(19) United States
(12) Patent Application Publication
(10) Pub. No.: US 2004/0052748 A1
(43) Pub. Date: Mar. 18, 2004

(54) Compositions of anionic polymeric rheology modifiers and cationic materials

(76) Inventor: Brian Jay Vondruska, Maple Heights, OH (US)

Correspondence Address: Noveon, Inc., Legal Department, 9911 Brecksville Road, Cleveland, OH, 44141-3247, US

(21) Appl. No.: 10/653,609

(22) Filed: Sep. 2, 2003

Related U.S. Application Data
(60) Provisional application No. 60/408,793, filed on Sep. 6, 2002.

(51) Int. Cl. A61K 7/06; A61K 7/11

(52) U.S. Cl. 424/70.12; 424/70.13

(57) ABSTRACT

A method of compatibilizing an anionic polymeric rheology modifier with cationic ingredients, which comprises complexing a cationic ingredient with an anionic complexing agent before combining the complexed cationic ingredient with an anionic rheology modifier. A composition comprising an anionic polymeric rheology modifier and a complexed cationic ingredient and a personal care or a household composition containing an anionic rheology modifier and a cationic ingredient complexed with an anionic complexing agent.

### REFERENCE TO RELATED APPLICATION

This application claims the benefit of U.S. Provisional Application Serial No. 60/408,793 filed Sep. 6, 2002, the disclosure of which is hereby incorporated by reference.

### BACKGROUND OF THE INVENTION

Rheology modifiers (thickeners) are generally employed in most personal care products and other products of that nature. Some of the most useful rheology modifiers are anionic polymeric materials that are based on ethylenically unsaturated carboxylic acid monomers which includes crosslinked polyacrylic acid or copolymers of ethylenically unsaturated carboxylic acid monomers and copolymerizable vinyl monomers. Such polymers yield anionic polymeric rheology modifiers that are extremely useful in various personal care products in the cosmetic and toiletry industries.

In addition to thickeners, such products generally require a variety of other ingredients especially cationic ingredients. Often cationic surfactants, or cationic conditioning agents, are particularly useful. However, cationic surfactants generally are not compatible with anionic polymeric thickening agents. G. Polotti and F. Coda in “Thickener for Cationic Surfactant Solutions” in the Proceedings of the 28th CED Annual Meeting, Barcelona, Spain, 1998, stated: “The thickening of cationic surfactant solutions is often a challenging problem in the detergent industry especially for the formulation of fabric softeners, toilet bowl cleaners, lime scale removers, etc. Part of the problem comes because the most common thickeners, such as those based on cross-linked polyacrylic acid, are anionic species. Although stable and viscous suspensions are achievable, the combination of polyacrylic acid and cationic surfactants forms aggregates that cannot be shared in further dilution. The effect of the cationic species is consequently lost in the strong bond with the anionic ingredients.”

In the Handbook of Cosmetic Science and Technology First Edition 1993 Elsevier Science Publishers Ltd, on page 17 it is stated:

“Carbomers are incompatible with cationic surfactants and show a significant reduction in viscosity building potential in the presence of electrolytes. For this reason, their use in the stabilization of detergent-based products is very limited.”

Consequently, there is a great need in the above-mentioned products for the ability to employ anionic polymeric thickeners or rheology modifiers such as carbomers in combination with cationic surfactants or other cationic ingredients.

There are several U.S. patents or published patent applications that disclose the use of rheology modifiers and silicones in various cosmetic or personal care compositions.

U.S. Pat. No. 4,210,161 discloses a cream rinse composition comprising an anionic polymer and a cationic surfactant capable of forming a water insoluble reaction product. Thus, this patent clearly states that the anionic polymer and a cationic surfactant are incompatible and do form a precipitate but in this formulation, such a precipitate is desirable.

U.S. Pat. No. 4,710,374 discloses cosmetic compositions containing a cationic polymer and an anionic polymer latex. The patent disclosure clearly stresses that the cationic polymer is of a relatively high molecular weight of between 500 to 3,000,000 but most, if not all, appear to be at least 10,000 molecular weight and more often, about 500,000 molecular weight. Thus, the cationic ingredient is a large molecule with a low charge density. For this reason, the cationic polymer and the anionic polymeric latex are not truly incompatible.

U.S. Pat. No. 6,071,499 discloses cosmetic compositions with an anionic acrylic polymer and an oxyalkylenated silicone which is nonionic. Since the silicone is not anionic, it cannot complex with a cationic ingredient although it is said to improve the performance of such anionic polymer.

Published U.S. application Ser. No. 2003/0,108,503 A1 discloses a composition comprising a copolymer of methacrylic acid and an alkyl acrylate, a cationic or amphoteric polymer and a functionalized silicone. Apparently, the disclosed anionic polymers are compatible with the disclosed cationic polymeric surfactants. The three components are combined together without first forming a complex of a cationic polymer with the functionalized silicone. Consequently, no compatibilization or complex formation is involved in the invention disclosed in this published application.

### SUMMARY OF THE INVENTION

The invention is directed to a method of compatibilizing an anionic polymeric rheology modifier with cationic ingredients, such as a cationic surfactant cationic polymer or a cationic salt, which method comprises complexing a cationic ingredient with an anionic complexing agent before combining the complexed cationic ingredient with an anionic rheology modifier. The invention is further directed to a composition comprising an anionic polymeric rheology modifier and a complexed cationic ingredient and to a personal care or a household composition containing an anionic rheology modifier and a cationic ingredient complexed with an anionic complexing agent.

When cationic ingredients are combined with anionic polymeric thickeners, because of their incompatibility, usually a precipitate forms, turbidity develops and the thickening effect of the polymers is generally substantially decreased. By first complexing the cationic ingredient(s) with an anionic complexing agent before combining with an anionic polymeric thickener, the incompatible anionic polymer thickeners and the cationic ingredients become compatibilized. When such compatibilized cationic ingredient(s) is combined with an anionic thickener, the viscosity/turbity profile of the resulting compositions is substantially improved. Thus, the complexing of the cationic materials prior to combining them with a thickener either reduced or eliminated excessive turbidity and the tendency to form precipitates.

### BRIEF DESCRIPTION OF THE DRAWINGS

FIGS. 1-3 are graphs showing compatibility of Carbopol® ETD 2020 thickener with various complexed cationics when a sufficient complexing agent is used.

FIGS. 4-6 are graphs showing compatibility of various Carbopol® thickeners with various cationics when complexing agents are used.

FIGS. 7-9 show the results of Rubine Dye tests.

FIGS. 10-11 show results of wet comb-through test results when a complex is formed and when the complexing agent is not used.

### DETAILED DISCLOSURE

The truly unexpected feature of the present invention is the fact that the cationic ingredients, which generally are not compatible with anionic polymeric thickening agents, can be made compatible by complexing them with anionic compatibilizing agents without negatively affecting the performance and function of the cationic ingredients. The cationic ingredients that may be used in personal care products in combination with anionic rheology modifiers are quaternary ammonium salts, polyquaternary ammonium salts, organic or inorganic salts, alkyl amines, amidoamines, ethoxylated amines and alkyl imidazolines which, as such, are incompatible with polymeric anionic rheology modifiers. By “incompatible”, is meant that when such cationic ingredients are combined with polymeric anionic rheology modifiers, either a precipitate forms or turbidity develops.

When cationic ingredients are added to a formulation containing an anionic thickening agent, generally a significant reduction in viscosity results and often a precipitate is formed and turbidity develops. For this reason, the use of anionic thickening agents in combination with cationic ingredients in personal care products and in household products is very limited. This long existing difficulty, however, can be overcome and such materials can be compatibilized by the instant invention, wherein cationic ingredients are first complexed with a compatibilizing agent which is an anionic bulky molecule containing an anionic group such as a sulfate group, sulfonate group, phosphate group, phosphonate or carboxylate groups. By “compatibilized” is meant a substantial reduction of the precipitate or turbidity that would be formed without first complexing the cationic ingredient. By “substantial reduction” is meant a reduction to such a degree that such ingredients (the cationic materials and the anionic thickeners) can be successfully employed in personal care products. Generally such reduction would constitute at least a 50% reduction of turbitity formation and preferably at least 80% reduction such that turbidity of compositions or formulations containing both cationic ingredient(s) and anionic rheology modifier(s) is not greater than 50, often 20 NTU and preferably 15 NTU or less. In clear gels, such as a clear conditioning styling gel, it is preferable that the turbidity be 15 NTU or less and preferably 10 NTU or less; while in a clear formula shampoo a turbidity of as high as 40 NTU may be acceptable. The level of turbidity that is considered acceptable always depends on the type of product. The use of complexed anionic ingredients of this invention also aid in efficient use of rheology modifiers by often enabling the use of a lesser amount of a thickener yet obtaining desirable properties, thus making the resulting products more cost efficient. There should be practically a complete elimination of precipitate formation.

Generally the cationic materials are not compatible with the anionic rheology modifiers. However, if the concentration of a cationic material is low enough, they may be compatible. Similarly, if the charge density is low enough (e.g. the charge moiety(s) is dispersed sparcely throughout the molecule) they may also be compatible. Consequently, this invention deals with cationic materials that are incompatible with the specified anionic polymeric rheology modifiers.

Cationic Ingredients

Cationic ingredients are commonly used in the personal care industry as surfactants and as conditioning ingredients. Since they are cationic in nature, it allows them to easily deposit onto anionic substrates like hair and skin.

Quaternary ammonium compounds (i.e. quats) are the most widely used of the many available classes of cationic ingredients which function as conditioning agents. Their outstanding performance characteristics, which greatly contribute to their popularity, are well-known in the industry. Their favorable safety profile, cost-effectiveness and long-term stability are additional factors.

Quats are used in hair care formulations (e.g. cleansing applications like shampoos, setting and fixing applications like mousses, gels, sprays, spritzes and volume enhancers, and coloring applications like one-part or two-part permanent or semi-permanent dyes) to enhance the shine, combability, appearance, body, slip, feel and general manageability of hair.

Polyquats are the polymeric counterparts of quats and are used in the same manner as quats, and for the same general purposes. They have additional utility as fixatives and rheology modifiers, due to their high molecular weight. Their large size also prevents them from penetrating (and thus, irritating) skin, so they enjoy market acceptance in skin care applications as well. In skin care, they are most commonly used as conditioners in personal cleansers like bath gels and body washes.

Illustrative examples of cationic ingredients are listed below.

A. Polyquaterniums

Hexadimethrine Chloride

Hydroxypropyl Guar Hydroxypropyltrimonium Chloride

Locust Bean Hydroxypropylthemonium Chloride

Polyacrylamidopropyltrimonium Chloride

Polymethacrylamidopropyltrimonium Methosulfate

Polyquaternium-1* to 20*, 22*, 24*, 27* to 37*, 39*, 42* to 50*

B. Monosubstituted Quaternaries

Hydroxypropyltrimonium Chloride

Basic Red 118*

Behenoyl PG-Trimonium Chloride

Behentrimonium Chloride

Behentrimonium Methosulfate

Benzyl Triethyl Ammonium Chloride

Bis-Hydroxyethyl Cocomonium Nitrate

Bis-Hydroxyethyl Dihydroxypropyl Stearammonium Chloride

Bis-Hydroxyethyl Rapeseedmonium Chloride

Bis-Hydroxyethyl Tallowmonium Chloride

Camphor Benzalkonium Methosulfate

Carpronium Chloride

Ceteartrimonium Chloride

Cetrimonium Bromide, Chloride, Methosulfate, Saccharinate and Tosylate

Cetyl Ethyldimonium Ethosulfate

Coco-Ethyldimonium Ethosulfate

Cocotrimonium Chloride and Methosulfate

C4-18 Perfluoralkylethyl Thiohydroxypropyltrimonium Chloride

Dextran Hydroxypropyltrimonium Chloride

Dimethicone Hydroxypropyl Trimonium Chloride

Dodecylbenzyltrimonium Chloride

Dodecylhexadecyltrimonium Chloride

Dodecylxylylditrimonium Chloride

Galactoarabinan Hydroxypropyltrimonium Chloride

Ginsing Hydroxpropyltrimonium Chloride

Guar Hydroxpropyltrimonium Chloride

Hydrogenated Tallowtrimonium Chloride

Hydroxypropyl Bistrimonium Diiodide

Hydroxypropyltrimonium Honey

Hydroxypropyltrimonium Hydrolyzed Whey

Isostearoyl PG-Trimonium Chloride

B. Monosubstituted Quaternaries (Cont'd)

Isostearyl Ethyidimonium Chloride

Lactamidopropyl Trimonium Chloride

Lauroyl PG-Trimonium Chloride

Laurtrimonium Bromide, Chloride and Trichlorophenoxide

Octyidodecyltrimonium Chloride

Oleamine Bishydroxypropyltrimonium Chloride

Oleoyl PG-Trimonium Chloride

Palmitamidopropyltrimonium Chloride

Palmitoyl PG-Trimonium Chloride

PEG-1 and PEG-10 Coco-Benzonium Chloride

PEG-2 and PEG-15 Cocomonium Chloride

PEG-5 Cocomonium Methosulfate

PEG-2 and PEG-15 Oleammonium Chloride

PEG-2 and PEG-15 Stearmonium Chloride

PEG-5 Stearyl Ammonium Chloride and Lactate

PEG-20 Tallow Ammonium Ethosulfate

PEG-5 Tallow Benzonium Chloride

PPG-9, PPG-25 and PPG-40 Diethylmonium Chloride

Quaternium-16*, 22*, 26*, 30*, 33*, 52*, 60*, 61*, 75* and 88*

Soytrimonium Chloride

Stearoyl PG-Trimonium Chloride

Steartrimonium Bromide

Steartrimonium Methosulfate

Steartrimonium Sacchannate

Tallow Trihydroxyethylammonium Acetate

Tallowtrimonium Chloride

C. Disubstituted Quaternaries

Behenalkonium Chloride

Benzalkonium Bromide and Chloride

Benthethonium Bromide or Chloride

Benzalkonium Cetyl Phosphate

Benzoxonium Chloride

C12-18 Dialkyldemonium Chloride

Cetalkonium Chloride

Cetearalkonium Bromide

Cetethyldimonium Bromide

Cetethyl Morpholinium Ethosulfate

Cetyl Pyrrolidonylmethyl Dimonium Chloride

Cocoalkonium Chloride

Denatonium Benzoate and Saccharide

Dibehenyl/Diarachidyl Dimonium Chloride

Dibehenyldimonium Chloride and Methosulfate

Di-C12-15, C12-18 and C14-18 Alkyl Dimonium Chloride

Dicetyldimonium Chloride

Dicocodimonium Chloride

Dicocoylethyl Hydroxyethylmonium Methosulfate

Didecyldimonium Chloride

C. Disubstituted Ouaternaries (Cont'd)

Dihydrogenated Palmoylethyl Hydroxyethylmonium Methosulfate

Dihydrogenated Palmoyl Hydroxyethylmonium Methosulfate

Dihydrogenated Tallow Benzylmonium Chloride and Hectorite

Dihydrogenated Tallowethyl Hydroxyethylmonium Methosulfate

Dihydrogenated Tallow Hydroxyethylominium Methosulfate

Dihydrogenated Tallow Hydroxyethylmonium Methosulfate

Dihydrogenated Tallowayethyl Hydroxyethylmonium Methosulfate

Dihydroxpropyl PEG-5 Linoleammonium Chloride

Diisostearamidopropyl Epoxypropylmonium Chloride

Dilaureth-4 Dimonium Chloride

Dilauryl Acetyl Dimonium Chloride

Dilauryldimonium Chloride

Dimethyl PABA Ethyl Cetearyldimonium Tosylate

Dimethyl PABA Midopropyl Laurdimonium Tosylate

Dioleoylamidoethyl Hydroxyethylmonium Methosulfate

Dioleoyl Edetolmonium Methosulfate

Dioleoyl EDTHP Monium Methosulfate

Dipalmitoylethyl Dimonium Chloride

Dipalmitoylethyl Hydroxyethylmonium Methosulfate

Dipalmoylethyl Hydroxyethylmonium Methosulfate

Dipalmoylisopropyl Dimonium Methosulfate

Disoydimonium Chloride

Disoyoylethyl Hydroxyethylmonium Methosulfate

Disteardimonium Hectorite

Disteareth-6 Dimonium Chloride

Distearoylethyl Dimonium Chloride

Distearoylethyl Hydroxyethylmonium Methosulfate

Distearoylpropyl Trimonium Chloride

Distearyldimonium Chloride

Distearyl Epoxypropylmonium Chloride

Ditallowamidoethyl Hydroxypropylmonium Methosulfate

Ditallow Dimonium Cellulose Sulfate

Ditallowdimonium Chloride

Ditallowoylethyl Hydroxyethylmonium Methosulfate

Ditridecyldimonium Chloride

Domiphen Bromide

Erucalkonium Chloride

Hydrogenated Tallowalkonium Chloride

Hydroxycetyl Hydroxyethyl Dimonium Chloride

Hydroxyethyl Cetyldimonium Chloride and Phosphate

Hydroxyethyl Laurdimonium Chloride

Hydroxyethyl Tallowdimonium Chloride

Hydroxypropyl Biscetearyldimonium Chloride

Hydroxypropyl Bisoleyldimonium Chloride

Hydroxypropyl Bisstearyldimonium Chloride

Isostearyl Laurdimonium Cloride

Lauralkonium Bromide and Chloride

Lauryl Methyl Gluceth-10 Hydroxypropyldimonium Chloride

C. Disubstituted Quatemaries (Cont'd)

Methylbenzethonium Chloride

Myristaklonium Chloride, Bromide and Saccharinate

Olealkonium Chloride

Oleoyl Epoxypropyldimonium Chloride

Panthenyl Hydroxypropyl Steardimonium Chloride

PEG-9 and 25 Diethylmonium Chloride

PEG-2 Dimeadowfoamamidoethylmonium Methosulfate

PEG-3 Dioleoylamidoethylmonium Methosulfate

PEG-5 Ditridecylmonium Chloride

PEG-8 Palmitoyl Methyl Diethonium Methosulfate

PEG-10 Stearyl Benzonium Chloride

PEG-3 Tallow Propylenedimonium Dimethosulfate

Quaternium-8*, 14*, 18*, 24*, 43*, 53*, 63*, 70*, 71* and 84*

Quaternium-18 Bentonite*

Quatemium-18 Benzalkonium Bentonite

Quaternium-18 Hectorite* and Methosulfate*

Sodium Coco PG-Dimonium Chloride Phosphate

Soy Dihydroxypropyldimonium Glucoside

Soydimonium Hydroxypropyl Hydrolyzed Wheat Protein

Soyethyldimonium Ethosulfate

Stearalkonium Bentonite, Chloride and Hectorite

Stearyl Ethylhexyldimonium Chloride and Methosulfate

Stearyl PG-Dimonium Chloride Phosphate

Tallowalkonium Chloride

Tallowdimonium Propyltrimonium Dichloride

Thiamine Diphosphate

D. Tetrasubstituted Quaternaries

Quaternium-15*

Tetrabutyl Ammonium Bromide

Tetramethylammonium Chloride

E. Heterocyclic Ouaternaries

Cetylpyridinium Chloride

Cocoyl Benzyl Hydroxyethyl Imidazolinium Chloride

Cocoyl Hydroxyethylimidazolinium PG-Chloride Phosphate

Dequalnium Acetate and Chloride

Dimethylaminostyrol Heptyl Methyl Thiazolium Iodide

Hydroxyanthraquinoneaminopropyl Methyl Morpholinium Methosulfate

Isostearyl Benzylimidonium Chloride

Isostearyl Ethylimidazolinium Ethosulfate

Lapyrium Chloride

Lauryl Isoquinolinium Bromide and Saccharinate

Laurylpyridinium Chloride

Platonin*

Quaternium-27*, 45*, 51*, 56*, 72*, 73*, 83* and 87*

Soyethyl Morpholinium Ethosulfate

Stearyl Hydroxyethylimidonium Chloride

Tall Oil Benzyl Hydroxyethyl Imidazolinium Chloride

F. Substituted Amido Quaternaries

Acetamidoethoxybutyl Trimonium Chloride

Acetamidopropyl Trimonium Chloride

Acrylamedopropyltrimonium Chloride/Acrylamide Copolymer

Acrylamidopropyltrimonium Chloride/Acrylates Copolymer

Almondamidopropalkonium Chloride

Apricotamidopropyl Ethyldimonium Ethosulfate

Avocadamidopropalkonium Chloride

Babassuamidopropalkonium Chloride

Behenamidopropyl Ethyldemonium Ethosulfate

Behenamidopropyl PG-Dimonium Chloride

Canolamridopropyl Ethyldiimonium Ethosulfate

Carboxymethyl Isostearamidopropyl Morpholine

Cinnamidopropyltrimonium Chloride

C14-20 and C18-22 Isoalkylamidopropylethyldimonium Ethosulfate

Cocamidopropyl Betaine MEA Chloride

Cocamidopropyldimonium Hydroxypropyl Hydrolyzed Collagen

Cocamidopropyl Ethyldimonium Ethosulfate

Cocamidopropyl PG-Dimonium Chloride and Chloride Phosphate

Cocamidopropyltrimonium Chloride

Dihydrogenated tallowamidoethyl Hydroxyethylmonium Chloride and Methosulfate

Hydroxypropyl Bisisostearamidopropyldimonium Chloride

Hydroxystearamidopropyl Trimonium Chloride

Hydroxystearamedopropyl Trimonium Methosulfate

Isononamidopropyl Ethyldimonium Ethosulfate

Isostearamidopropyl Epoxypropyl Dimonium Chloride

Isostearamidopropyl Epoxypropylmorpholinium Chloride

Isostearamidopropyl Ethyldimonium Ethosulfate

Isostearamidopropyl Ethylmorpholinium Ethosulfate

Isostearamidopropyl Laurylacetodimonium Chloride

Isostearamidopropyl PG-Dimonium Chloride

Isostearamenopropalkonium Chloride

Isostearyl Behenamidopropyl Betainate

Isostearyl Dilinoleamidopropyl Betainate

Isostearyl Racinoleamidopropyl Betainate

Methylene bis (tallowacetamiddimonium Chloride)

Lauramidopropyl Acetamidodimonium Chloride

Lauramidopropyl PG-Dimonium Chloride

Linoleamidopropyl Ethyldimonium Ethosulfate

Linoleamidopropyl PG-Dimonium Chloride Phosphate and Phosphate Dimethicone

Minkamidopropalkonium Chloride

Minkamidopropyl Ethyldimonium Ethosulfate

Oleamidopropyldimonium Hydroxypropyl Hydrolyzed Collagen

Oleamidopropyl Ethyldimonium Ethosulfate

Oleamidopropyl PG-Dimonium Chloride

Rapeseedamidopropyl Benzyldimonium Chloride

Rapeseedamidopropyl Epoxypropyl Dimonium Chloride

F. Substituted Amido Quaternaries (Con'd)

Rapeseedamidopropyl Ethyldimonium Ethosulfate

Ricebranamidopropyl Hydroxyethyl Dimonium Chloride

Ricinoleamidopropyl Ethyldimonium Ethosulfate

Ricinoleamidopropyltrimonium Chloride and Methosulfate

Saffloweramidopropyl Ethyldimonium Ethosulfate

Sodium Borageamidopropyl PG-Dimonium Chloride Phosphate

Sodium Emuamidopropyl PG-Dimonium Chloride Phosphate

Sodium Milkamidopropyl PG-Dimonium Chloride Phosphate

Sodium Oleamidopropyl PG-Dimonium Chloride Phosphate

Sodium Sunfloweramidopropyl PG-Dimonium Chloride Phosphate

Soyamidoethyldimonium/Trimonium Hydroxypropyl Hydrolyzed Wheat Protein

Soyamidopropalkonium Chloride

Soyamidopropyl Ethyldimonium Ethosulfate

Stearamidopropalkonium Chloride

Stearamidopropyl Cetearyl Dimonium Tosylate

Stearamidopropyl Ethyldimonium Ethosulfate

Stearamidopropyl PG-Dimonium Chloride Phosphate

Stearamidopropyl Pyrrolidonylmethyl Dimonium Chloride

Stearamidopropyl Trimonium Methosulfate

Undecylenamidopropyltrimonium Methosulfate

Wheat Germamidopropalkonium Chloride

Wheat Germamidopropalkonium Hydroxypropyl Hydrolyzed Wheat Protein

Wheat Germamidopropyl Epoxypropyldimonium Chloride

Wheat Germamidopropyl Ethyldimonium Ethosulfate

G. Quaternized Keratin

AMP-Isostearolyl Gelatin/Keratin Amino Acids/Lysine

Cocodimonium Hydroxypropyl Hydrolyzed Hair Keratin and Keratin

Hydroxypropyltrimonium Gelatin and Hydrolyzed Keratin

Lauryldimonium Hydroxypropyl Hydrolyzed Keratin

Quaternium-79 Hydrolyzed Keratin*

Steardimonium Hydroxypropyl Hydrolyzed Keratin

H. Quaternized Collagen

Benzyltrimonium Hydrolyzed Collagen

Cocodimonium Hydroxypropyl Hydrolyzed Collagen

Hydroxypropyltrimonium Hydrolyzed Collagen

Lauryldimonium Hydroxypropyl Hydrolyzed Collagen

Propyltrimonium Hydrolyzed Collagen

Quaternium-76 and 79 Hydrolyzed Collagen*

Steardimonium Hydroxypropyl Hydrolyzed Collagen

Steartrimonium Hydroxyethyl Hydrolyzed Collagen

Triethonium Hydrolyzed Collagen Ethosulfate

I. Quaternized Amino Acids

Cocodimonium Hydroxypropyl Silk Amino Acids

Gelatin/Keratin Amino Acids/Lysine Hydroxypropyltrimonium Chloride

J. Quatemized Proteins

Cocodimonium Hydroxypropyl Hydrolyzed Casein, Silk, Rice Protein, Soy Protein & Wheat Protein

Gelatin/Lysine/Polyacrylamide Hydroxypropyltrimonium Chloride

Hydroxypropyltrimonium Hydrolyzed Casein and Conchiolin Protein

Hydroxypropyltrimonium Hydrolyzed Rice Bran Protein, Silk, Vegetable Protein,

Wheat Protein, Wheat Protein/Siloxysilcate

Laurdimonium Hydroxypropyl Hydrolyzed Soy Protein and Wheat Protein/Siloxysilicate

Lauryldimonium Hydroxypropyl Hydrolyzed Casein, Silk and Soy Protein

Propyltrimonium Hydrolyzed Soy Protein and Wheat Protein

Quatemium-79 Hydrolyzed Milk Protein*, Silk*, Soy Protein* and Wheat Protein*

Quaternium-86*

Steardimonium Hydroxypropyl Hydrolyzed Casein, Rice Protein, Silk, Soy Protein and Vegetable Protein

Steardimonium Hydroxypropyl Wheat Protein

K. Salts of Divalent or Polyvalent Cations

Aluminum Acetate and Acetate Solution

Aluminum Benzoate, Butoxide, Citrate, Diacetate, Dicetyl Phosphate, Lactate,

Methionate, PCA, Sucrose Octasulfate and Triformate

Aluminum/Magnesium Hydroxide Stearate

Antimony Potassium Tartrate

Barium Gluconate

Bismuth Citrate and Subgallate

Brucine Sulfate

Calcium Acetate, Ascorbate, Benzoate, Citrate, Cyclamate, DNA, Fructoheptonate, Glucoheptonate, Gluconate, Glycerophosphate, Lactate, Pantetheine Sulfonate,Pantothenate, Paraben, Propionate, Saccharine, Salicylate, Sorbate, Stearoyl Lactylate, Tartarate and Thioglycolate

Calcium Disodium EDTA

Cobalt Gluconate

Copper DNA, Gluconate, PCA, PCA Methylsilanol, Picolinate and Usnate

Cupric Acetate

Feric Ammonium Citrate

Ferric Citrate and Glycerophosphate

Ferrous Aspartate, Aglucoheptonate and Gluconate

Iron Picolinate

Isopropyl Titanium Triisostearate

Lead Acetate

Magnesium Acetate, Ascorbate, Ascorbate/PCA, Ascorbyl Phosphate, Benzoate, Citrate, DNA, Glucohiptonate, Gluconate, Glyerophosphate, PCA, Propionate, Salicylate and Thioglycolate

Magnesium Laureth-11 Carboxylate

Manganese Gluconate

Manganese Glycerophosphate

Manganese PCA

Molybdenum Aspartate

K. Salts of Divalent or Polyvalent Cations (Cont'd)

Nickel Gluconate

Phenyl Mercuric Acetate, Benzoate, Borate and Chloride

Strontium Acetate

Strontium Thioglycolate

Zinc Acetate, Citrate, Cysteinate, Dibutyldithiocarbamate, Glucoheptonate, Gluconate, Glycyrrhelinate, Lactate, Picolinate and Pyrithione

Zinc Formaldehyde Sulfoxylate

Zinc PCA

L. Pigments

Zinc Oxide, Iron Oxides, Titanium Dioxide

M. Organic Amines

Alanine Glutamate

Allantoin Acetyl Methionine, Ascorbate, Biotin, Calcium Pantothenate, Galacturonic Acid, Glycyrrhetinic Acid, PABA and Polygalacturonic Acid

Amodimethicone Hydroxystearate

Arginine Aspartate, DNA and PCA

Arginine Glutamate

Arginine Hexyldecyl Phosphate

Chitosan Adipate, Ascorbate, Glycolate and Salicylate

Chloramine T

Chlorhexidine Diacetate, Digluconate and Dihydrochloride

Chlorophyllin-Copper Complex

Ciclopirox Olamine

Cysteamine HCI

Cysteine DNA

DEA-Cetyl Phosphate

DEA-Hydrolyzed Lecithin

DEA-Methoxycinnamate

Dibehenamidopropyldimethylamine Dilinoleate

Dibromopropamidine Diisethionate

Diglycol Guanidine Succinate

Dihydroxyethyl Tallowamine Oleate

Dilithium Oxalate

Dimethicone Propylethylenediamine Behenate

Ethanolamine Dithiodiglycolate, Glycerophosphate and Thioglycolate

Ethyl Hydroxy Picolinium Lactate

Ethyl Lauroyl Arginate HCI

Guanidine Carbonate, HCI and Phosphate

Hexamidine Diisethionate and Paraben

Isostearamidopropyl Dimethylamine Gluconate, Glycolate and Lactate

Isostearamidopropyl Morpholine Lactate

Lauryl Isoquinolinium Saccharinate

Lauryl PCA

Lysine DNA and Glutamate

MEA-Benzoate, Dicetearyl Phosphate, o-Phenylphenate, Salicylate, Thiolactate and Undecylenate

MEA-Laureth-6 Carboxylate

M. Organic Amines (Cont'd)

MEA PPG-6 Laureth-7 Carboxylate

MEA PPG-8 Steareth-7 Carboxylate

Methyl Hydroxycetyl Glucaminium Lactate

Methylsilanol Hydroxyproline Aspartate

Nicotinyl Tartrate

Olivamidopropyl Dimethylamine Lactate

Oxyquinoline Benzoate and Sulfate

PCA Ethyl Cocoyl Arginate

Piroctone Olamine

Pyridoxine HCI

Saccharated Lime

TEA-Cocoyl Alaninate

TEA-EDTA

TEA-Lauroyl Lactylate

TEA-Phenylbenzimidazole Sulfonate

Thurfylnicotinate HCI

N. Organic Imidazolines

Stearyl Hydroxyethyl Imidazoline

O. Ethoxylated Amines

PEG-1 5 Tallowamine

PEG-cocopolyamine

P. Quanternized Cellulose

PG-Hydroxyethylcellulose Cocodimonium Chloride

PG-Hydroxyethylcellulose Lauryldimonium Chloride

PG-Hydroxyethylcellulose Stearyldimonium Chloride

Q. Quatemized Silicone

Quaternium-80*

Silicone Quaternium-1* to 13*

R. Multifunctional Quaternaries

Quaternium-77*, 78*, 81*, 82* and 85*

S. Tertiary Substituted Quaternaries

Tricetylmonium Chloride *The composition of this material is identified in the International Cosmetic Ingredients Dictionary and Handbook, 8th ed. (2000), the Cosmetic Toiletry and Fragrance Association, 1101 17th St., NW, Suite 300, Washington, D.C. 20036-4702.

Compatibilizint Agents

The compatibilizing agents or complexing agents which complex with the cationic ingredients may be any material that contains a “bulky” molecule having an anionic group. The “bulky” molecule should not be reactive chemically with either the anionic thickening agent or the cationic ingredients. The “bulky” molecule will generally have a molecular weight of at least 500 Mn, preferably at least 1,000 Mn, and may have a molecular weight of up to 50,000 Mn, but generally up to 25,000 Mn. Usually the “bulky” molecule is a polymeric material having at least three repeat units. The composition of the polymeric materials may be heterogeneous and predominantly may be polysilicones, acrylic copolymers, polyalkylene glycol such as polyethylene glycol and polypropylene glycol, polyvinyl alcohol, polyvinyl acetate, polysaccharide such as starch and cellulose or polyurethane. Polyalkylene glycols may contain terminal groups such as, but not limited, allyl, propenyl, propyl and hydrogen or others. These polymeric or “bulky” groups must contain anionic groups which will complex with the cationic ingredients. The preferred anionic groups are carboxylate (—COOH), sulfonate (—SO₃H), sulfate (—OSO₃H), phosphate (—OP(OH)₂) and phosphonate (—PO(OH)₂). The anionic groups complex with the cationic ingredients preventing the cationic ingredients from interfering with the anionic thickening agent and permitting the thickening agent to perform its viscosity building function. Although, in principle, any polymeric material containing anionic groups may be employed, it is preferable to employ silicones because they also serve to condition keratinous substances such as hair in shampoos, hair rinses, hair gels and hair dyes; or skin in lotions, creams and hand sanitizers; or nails in nail strengtheners or coatings and cuticle softeners; or lips in lipsticks, lip balms and the like.

The preferred silicone complexing agents may be represented generically

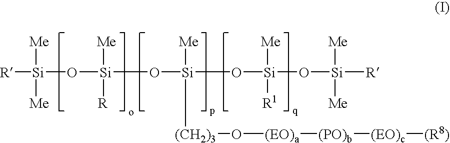

wherein:

Me is methyl;

R and R′ are independently selected from methyl, —OH, —R⁷, and —R⁹-A or —(CH₂)₃—O-(EO)ₐ—(PO)_{b}-(EO)_{c}-G with the proviso that both R and R′ are not methyl, —OH or R⁷;

R¹ is selected from lower alkyl CH₃(CH₂)ₙ— or phenyl where n is an integer from 0 to 22;

a, b, and c are integers independently ranging from 0 to 100;

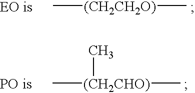

o is an integer ranging from 1 to 200;

q is an integer ranging from 0 to 1000;

p is an integer ranging from 0 to 200;

R⁷ is aryl, alkyl, aralkyl, alkaryl, or alkenyl group of 1-40 carbons;

R⁸ is hydrogen or R⁷ or C(O)—X wherein X is aryl, alkyl, aralkyl, alkaryl, alkenyl group of 1-40 carbons, or a mixture thereof;

R⁹ is divalent group selected from alkylene of 1-40 carbons which may be interrupted with arylene group of 6 to 18 carbons or an alkylene group containing unsaturation of 2 to 8 carbons;

A and G are independently are selected from

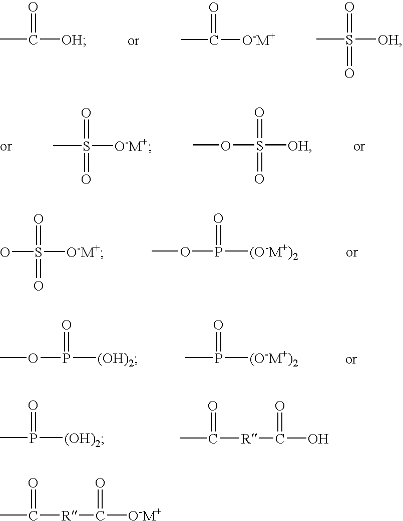

where

R″ is a divalent group selected from alkylene of 1-40 carbons which may be interrupted with an arylene group of 6 to 18 carbons or an alkylene group of 2 to 8 carbons, and is preferably selected from the

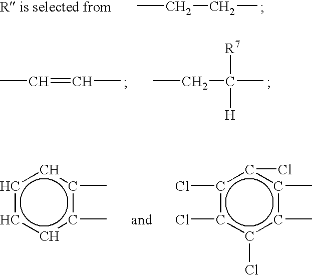

where M is Na, K, Li, NH₄; or an amine containing alkyl, aryl, akenyl, hydroxyalkyl, arylalkyl or alkaryl groups.

Another category of silicone complexing agents is silicone sulfates which may be represented by the following formula:

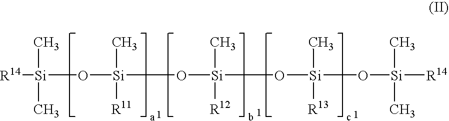

wherein

R¹¹ is selected from lower alkyl having one to eight carbon atoms or phenyl,

R¹² is

—(CH₂)₃—O-(EO)ₓ—(PO)_{y}-(EO)_{z}—SO₃⁻M⁺

M is a cation and is selected from Na, K, Li, or NH₄;

x, y and z are integers independently ranging from 0 to 100;

R¹³ is

—(CH₂)₃—O-(EO)ₓ—(PO)_{y}-(EO)_{z}—H

R¹⁴ is methyl or hydroxyl;

a¹ and c¹ are independently integers ranging from 0 to 50;

b¹ is an integer ranging from 1 to 50;

A still further category of silicone complexing agents may be represented as follows:

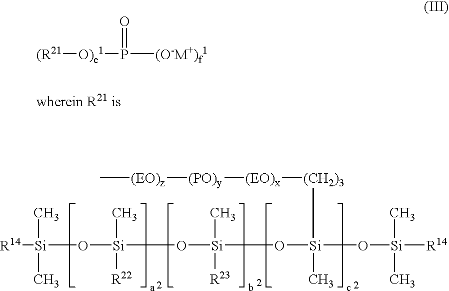

a¹ is an integer from 0 to 200;

b² is an integer from 0 to 200;

c² is an integer from 1 to 200;

R¹⁴ is as defined above;

R²² is selected from —(CH₂)ₙCH₃ and phenyl;

n is an integer from 0 to 10;

R²³ is —(CH₂)₃—O-(EO)ₓ₁—(PO)_{y1}-(EO)_{z1}—H;

x¹, y¹ ands z¹ are integers and are independently selected from 0 to 20;

e¹ and f¹ are 1 or 2 with the proviso that e+f=3;

M is selected from H, Na, K, Li, or NH₄; and

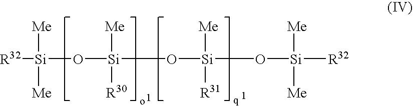

wherein;

Me is methyl;

R³⁰ and R³² independently are CH₃ or

—(CH₂)₃—O-(EO)ₐ_{³}—(PO)_{b}_{³}-(EO)_{c}_{³}—C(O)—R³³—C(O)—OH;

with the proviso that both R³⁰ and R³² are not —CH₃;

R³³ is selected from —CH₂—CH₂—; —CH═CH—; —CH₂—C(R³⁷)—H;

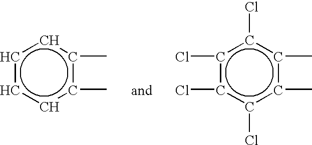

R³⁷ is alkyl having from 1 to 22 carbon atoms;

R³¹ is selected from lower alkyl (having 1-4 carbons), CH₃(CH)ₙ¹— and phenyl;

n¹ is an integer from 0 to 8;

a³, b³ and c³ are integers independently ranging from 0 to 20;

EO is an ethylene oxide residue —(CH₂CH₂—O)—;

PO is a propylene oxide residue —(CH₂CH(CH₃)—O);

o¹ is an integer ranging from 1 to 200;

q¹ is an integer ranging from 0 to 500.

It should be noted that in the above structure units EO and PO may be in random and block structures.

Such silicone carboxylates are disclosed in greater detail in U.S. Pat. No. 5,296,625, the disclosure of which is incorporated herein by reference. Still further silicone complexing agents are silicones containing a multiplicity of different anionic substituents. Such silicones can be prepared by reacting two or more types of anionic silicones already disclosed using reactions well known to those in the art. The resulting molecule could be a hybrid of the starting silicones and would, therefore, contain multiple types of anionic functional groups. The properties of the silicone can be optimized in such a fashion. One type of reaction, the silicone equilibration reaction, involves charging a reactor with raw materials, adding a suitable catalyst, mixing with heat, and then neutralizing the catalyst. The Chemistry is discussed in Silicone in Organic, Organometallic and Polymer Chemistry (Michael Brook)—John Wiley and Sons, New York, 2000, pp. 261-266.

The amount of the anionic complexing agent required to complex the cationic ingredients will depend on the specific cationic ingredients (the quat, polyquat, organic salt, etc.), the amount of the cationic ingredients present and the overall pH of the final formulation. The lower the pH of the final formulation, the greater the amount of the complexing agent is required. In view of the above-mentioned variables, it will be necessary to conduct some routine testing to arrive at the optimum amount of the anionic complexing agent, such as a silicone, to be used in a particular formulation to provide the desired results. Generally, the weight ratio of the anionic complexing agent, such as the anionic silicone complexing agent, to the cationic ingredient or ingredients, will be in the range of 0.1-10 to 1. Preferably, the weight ratio of the complexing agent to the cationic ingredient(s) will be 0.5-6 to 1 and most preferably 1.5-3 to 1.

Anionic Polymeric Rheology Modifiers

The polymeric rheology modifiers (thickening agents) that normally are not compatible with cationic ingredients, may be used in various formulations in combination with complexed cationic ingredients. Therefore, anionic polymeric rheology modifiers may be employed in the compositions of this invention.

Generally such anionic polymeric rheology modifiers are either homopolymers obtained from ethylenically unsaturated monomers containing carboxylic groups or ethylenically unsaturated monomers derived from those that contain carboxylic groups, such as acid hydrides, anhydrides or esters. These include the homopolymers of such carboxylic group containing monomers or ethylenically unsaturated anhydrides or copolymers containing at least 1% by weight of such carboxylic monomers or anhydride monomers, preferably at least 5% and more preferably at least 10%. Prior art discloses a variety of such homopolymers and copolymers that are useful as thickening agents. Illustrative examples of such thickening agents are discussed below.

The carboxylic monomers useful in the production of thickener polymers are the olefinically-unsaturated carboxylic acids containing at least one activated carbon-to-carbon olefinic double bond, and at least one carboxyl group, that is, an acid containing an olefinic double bond which readily functions in polymerization because of its presence in the monomer molecule either in the alpha-beta position with respect to a carboxyl group thusly,

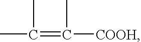

or as a part of a terminal methylene grouping thusly, CH₂═C<. In the alpha-beta acids the close proximity of the strongly polar carboxyl group to the double-bonded carbon atoms has a strong activating influence rendering the substances containing this structure very readily polymerizable. The presence of a terminal methylene grouping in a carboxylic monomer makes this type of compound much more easily polymerizable than if the double bond were intermediate in the carbon structure. Olefinically-unsaturated acids of this class include such widely divergent materials as the acrylic acids typified by acrylic acid itself, methacrylic acid, ethacrylic acid, alpha-chloroacrylic acid, alpha-cyano acrylic acid, beta methyl-acrylic acid (crotonic acid), alpha-phenyl acrylic acid, beta-acryloxy propionic acid, sorbic acid, alpha-chloro sorbic acid, angelic acid, cinnamic acid, p-chloro cinnamic acid, beta-styryl acrylic acid (1-carboxy-4-phenyl butadiene-1,3), itaconic acid, citraconic acid, messaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same polycarboxylic acid molecule. Anhydrides of the types formed by elimination of water from two or more molecules of the same or different unsaturated acids, such as acrylic anhydride, are not included because of the strong tendency of their polymers to hydrolyze in water and alkali. Maleic anhydride and the other acid anhydrides useful herein have the general structure

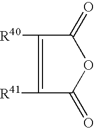

wherein R⁴⁰ and R⁴¹ are independently selected from the group consisting of hydrogen, cyanogens (—C≡N), hydroxyl, lactam and lactone groups and alkyl, aryl, alkaryl, aralkyl, and cycloalkyl groups such as methyl, ethyl, propyl, octyl, decyl, phenyl, tolyl, xylyl, benzyl, cyclohexyl and the like.

The preferred carboxylic monomers for use in this invention are the monoolefinic acrylic acids having the general structure

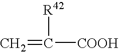

wherein R⁴² is a substituent selected from the class consisting of hydrogen, halogen, hydroxyl, lactone, lactam cyanogen (—CN), monovalent alkyl group (1 to 4 carbons), monovalent aryl group (6 to 12 carbons), monovalent aralkyl group (7 to 12 carbons), monovalent alkaryl group (7 to 12 carbons) and monovalent cycloaliphatic group (4 to 8 carbons). Of this class, acrylic acid itself is most preferred because of its generally lower cost, ready availability, and ability to form superior polymers. Another particularly preferred carboxylic monomer is maleic anhydride.

The preferred acrylic ester monomers having long chain aliphatic groups are derivatives of acrylic acid represented by the formula:

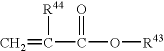

wherein R⁴³ is hydrogen or an alkyl group having from 8 to 30 carbon atoms, preferably 10 to 22 carbon atoms and R⁴⁴ is hydrogen or a methyl group. Representative higher alkyl acrylic esters are decyl acrylate, lauryl acrylate, stearyl acrylate, behenyl acrylate and melissyl acrylate and the corresponding methacrylates. Mixtures of two or three or more long chain acrylic esters may be successfully polymerized with one of the carboxylic monomers to provide useful thickening resins of this invention.

The preferred crosslinking monomer, if one is employed, is a polyalkenyl polyether having more than one alkenyl ether grouping per molecule. The most useful possess alkenyl groups in which an olefinic double bond is present attached to a terminal methylene grouping, CH₂═C<. They are made by the etherification of a polyhydric alcohol containing at least 4 carbon atoms and at least 3 hydroxyl groups. Compounds of this class may be produced by reacting an alkenyl halide, such as allyl chloride or allyl bromide with a strongly alkaline aqueous solution of one or more polyhydric alcohols. The product is a complex mixture of polyethers with varying numers of ether groups. Analysis reveals only the average number of ether groupings on each molecule. Efficiency of the polyether crosslinking agent increases with the number of potentially polymerizable groups on the molecule. It is preferred to utilize polyethers containing an average of two or more alkenyl ether groupings per molecule. Other crosslinking monomers include for example, diallyl esters, dimethallyl ethers, allyl or menthally acrylates and acrylamides, tetraallyl tin, tetravinyl silane, polyalkenyl methanes, diacrylates and dimethacrylates, divinyl compounds, polyallyl phosphate, diallyloxy compounds and phosphite esters and the like.

Monomeric mixtures of the carboxylic monomer and the long chain acrylic ester monomer preferably contain 95 to 50 weight percent carboxylic monomer and 5 to 50 weight percent acrylic ester monomer.

The above-discussed polymeric thickening agents are disclosed in greater detail in U.S. Pat. No. 3,940,351, the disclosure of which is incorporated herein by reference. Related polymeric thickeners are disclosed in U.S. Pat. No. 3,915,921, the disclosure of which is also incorporated herein by reference.

Another class of thickeners is represented by crosslinked copolymers obtainable by copolymerization of a monomeric system comprising:

a) from about 10 to about 97% by weight of at least one ethylenically unsaturated mono- or dicarboxylic acid;

b) from 0 to about 80% by weight of at least one (C₁₋₃₀) alkyl or aralkyl ester of an ethylenically unsaturated mono- or dicarboxylic acid;

c) from about 0.5 to about 80% by weight of at least one associative monomer which is an ester of formula

J-O—(CH₂—CHR₂O)ᵣ—(CH₂)ₛ—R₁

wherein

J is an ethylenically unsaturated acylic residue, optionally containing an additional carboxylic group, wherein, optionally, said additional carboxylic group may be esterified with a (C₁₋C₂₀) aliphatic alkyl group;

R₁ is an alkyl, alkylphenyl or aralkyl residue having from 1 to 30 carbon atoms;

R₂ is hydrogen, methyl or ethyl;

r is comprised between 0 and 50;

s is comprised between 0 and 30;

d) from 0 to about 20% by weight of at least one ethylenically unsaturated amide;

e) from about 0.2 to about 20% by weight of at least one diester between a polyoxyalkyleneglycol or an emulsifier having at least two free OH-groups and an ethylenically unsaturated carboxylic acid, as the cross-linking agent;

f) from 0 to about 20% by weight of at least one ethylenically unsaturated sulfonic acid.

Examples of ethylenically unsaturated mono- or dicarboxylic acids as indicated under a) are, for example, acrylic, methacrylic, itaconic, maleic, sorbic, crotonic acids, and analogs. Among these, acrylic and methacrylic acids are the preferred ones.

Preferred esters of ethylenically unsaturated mono- or dicarboxylic acids indicated under b) are methyl acrylate, ethyl acrylate, methyl methacrylate, butyl acrylate, ethyl methacrylate and analogs. The most preferred ones are methyl and ethyl (meth)acrylate.

The associative monomer c) may be any compound falling within the above formula J-O—(CH₂—CHR₂O)ᵣ—(CH₂)ₛ—R₁ wherein R₁ and R₂ are as above indicated, the sum of r and s may vary between 0 and 80 and J is the acrylic residue of an ethylenically unsaturated acid selected from acrylic, methacrylic, itaconic, maleic, sorbic, crotonic, oleic and linoleic acids. Preferred are the esters of cetylstearylalcohol ethoxylated with 25 moles of ethylene oxide. The associative monomers c) are commercially available products, or they can be prepared substantially according to procedures known in the art (U.S. Pat. Nos. 3,652,497 and 4,075,411).

The preferred ethylenically unsaturated amides d) are acrylamide, methacrylamide and vinylpyrrolidone, whereas the preferred ethylenically unsaturated sulfonic acids f) are vinylsulfonic acid and p-styrenesulfonic acid.

The crosslinking agents listed under point e) above can have one of the following structures of formula (I), (II) or (IV), or they are polyethoxylated derivatives of castor oil, optionally hydrogenated in whole or in part, esterified with ethylenically unsaturated carboxylic acids, with the proviso that the total number of ethylenic bonds is at least two.

The cross-linking agent e) is a compound of formula (I):

D₁-O—(CH₂—CHZ₁-O)ₜ—(CH₂—CHZ₂-O—)ᵤ—(CH₂—CHZ₃-O)_{w}-D₂ (I)

wherein:

D₁ and D₂, which can be the same or different, are an ethylenically unsaturated acylic residue, which may contain an additional carboxylic group wherein, optionally, said additional carboxylic group can be esterified with a (C₁₋₂₀) aliphatic alkyl group;

Z₁ and Z₃ represent independently hydrogen or an (C₁₋₂₀) aliphatic alkyl or aralkyl group;

Z₂ is hydrogen or methyl;

t and w are integers comprised between 0 and 20;

u is an integer comprised between 1 and 100;

the sum t+u+w may represent any integer comprised between 1 and 140;

with the proviso that, when Z₁, Z₂ and Z₃ are simultaneously hydrogen and D₁ and D₂ are simultaneously the acryl residue of methacrylic acid, the sum t+u+w cannot be 1;

and wherein the structure of the polyalkyleneglycol may be random or block.

Preferably, in the crosslinking agents of formula (I), D₁ and D₂ represent, independently, the acylic residue of acrylic, methacrylic, itaconic, maleic, sorbic, crotonic, oleic or linoleic acid, Z₁, Z₂ and Z₃ represent hydrogen or methyl, the sum a+b+c is higher than 10 and the structure of the polyalkyleneglycol may be random or block.

More preferably, in the crosslinking agents of formula (I), D₁ and D₂ represent, independently, the acylic residue of acrylic, methacrylic or itaconic acid, Z₁, Z₂ and Z₃ represent hydrogen, and the sum t+u+w is higher than 20.

The crosslinking agents of formula (I) are products deriving from the esterification of polyalkyleneglycols with ethylenically unsaturated carboxylic acids; some of them are described in the literature (U.S. Pat. Nos. 3,639,459 and 4,138,381; DD Patent 205,891; Polymer, 1978, 19(9), 1067-1073; Pigm. Resin. Technol., 1992, 21(5), 16-17).

The compounds of formula (I) can also be prepared by esterification of the compounds of formula (Ia)

H—O—(CH₂—CHZ-O)ₜ—(CH₂—CHZ₂-O—)ᵤ—(CH₂CHZ₃-O)_{w}—H (1a)

wherein, Z₁, Z₂, Z₃, t, u and w are as above defined, with a carboxylic acid D₁-OH and/or D₂-OH, wherein D₁ and D₂ are as above defined, or the corresponding anhydride or acyl halide or, alternatively, by trans-esterification of the corresponding esters of low-boiling alcohols.

The crosslinking agent (e) is a compound of formula (II)

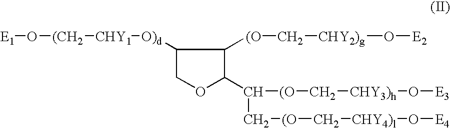

wherein:

E₁, E₂, E₃ and E₄ represent independently hydrogen or the acylic residue of a saturated or ethylenically unsaturated mono- or dicarboxylic acid from 2 to 25 carbon atoms, in which the further carboxylic group can optionally be esterified with a (C₁₋₂₀) aliphatic alkyl group, with the proviso that at least two of E₁, E₂, E₃ and E₄ represent ethylenically unsaturated acylic residues as above defined;

Y₁, Y₂, Y₃ and Y₄, which can be the same or different, are hydrogen, methyl or ethyl;

d, g, h and i are integers comprised between 0 and 30.

Preferably, the compounds of formula (II) are sorbitan derivatives (all of d, g, h and i are 0) or sorbitan derivatives ethoxylated with from about 4 to about 20 moles of ethylene oxide, in which at least two of the hydroxy groups are esterified with ethylenically unsaturated carboxylic acids selected from acrylic, methacrylic, itaconic, maleic, sorbic, crotonic, oleic and linoleic acids, and at least one of the two residual hydroxy groups is esterified with a fatty acid from 10 to carbon atoms.

The compounds of formula (II) are prepared by introducing the ethylenically unsaturated acyl groups as reported above in the preparation of the compounds of formula (I). The starting substrate is a compound of formula (II) wherein at least two of E₁, E₂, E₃ and E₄ represent hydrogen, and the remaining of E₁, E₂, E₃ and E₄ can be hydrogen or an acyl group as above defined.

The cross-linking agent e) may further be a polyethxoxylated derivative of castor oil, optionally partially or totally hydrogenated, esterified with an ethylenically unsaturated carboxylic acid, with the proviso that, in said cross-linking agent, the total number of bonds of ethylenic type is at least two. Preferred are the polyethoxylated derivatives of castor oil with an ethoxylation degree varying from about 15 to about 150, esterified with acids selected from acrylic, methacrylic, itaconic, maleic, sorbic, crotonic, oleic and linoleic acids.

These compounds are prepared by esterification of the corresponding polyethxoxylated derivatives of castor oil, optionally partially or totally hydrogenated, following procedures known in the art.

The crosslinking agent e) may be a compound of formula (IV)

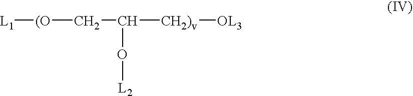

wherein:

L₁, L₂ and L₃, which may be the same or different, are hydrogen or an acyl residue of a saturated or unsaturated mono- or dicarboxylic acid from 2 to 25 carbon atoms, in which the further carboxylic group can optionally be esterified with a (C₁₋₂₀)aliphatic alkyl group, with the proviso that at least two of L₁, L₂ and L₃ represent an ethylenically unsaturated acylic residue as above defined;

p is an integer comprised between 2 and 50.

Also the compounds of formula (IV) are prepared through the above-illustrated conventional procedures, starting from a polyglycerol of formula

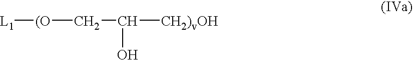

The cross-linked copolymers of the invention can be prepared by different polymerization procedures such as, for instance, the precipitation polymerization, suspension and solution polymerizations, or the emulsion polymerizations of the type oil-in-water or water-in-oil. The conditions of the polymerization reactions are, basically, those known in the art. Generally, the polymerizations are performed in the presence of anionic surfactants/emulsifiers, such as, for instance, sodium dodecylbenzenesulfonate, sodium disecondary-butylnaphthalene sulfonate, sodium laurylsulfate, sodium laurylether sulfate, disodium dodecyldiphenyl ether disulphonate, disodium n-octadecylsulfo-succinamate or sodium dioctylsulfosuccinate. Particularly preferred are sodium laurylsulfate and sodium laurylether sulfate. The temperature is generally comprised between about 50 and about 120° C., and the polymerization is completed in about 2-8 hours. The most preferred polymerization reaction is the oil-in-water emulsion polymerization.

The above-discussed class of thickeners are disclosed in greater detail in U.S. Pat. No. 6,140,435 which disclosure is incorporated herein by reference.

Anionic polymeric rheology modifiers or thickening agents are available commercially from many suppliers under a variety of trade names. Thus, Noveon, Inc. (formerly The B.F. Goodrich Company) sells Carbopol® thickener resins in a variety of grades and products for various uses and applications. 3V/Sigma supplies a series of thickener products under the Synthalen® series, Stabylen®, PNC® and Polygel®. Rita sells the Acritamer® series of products. Pomponesco sells Addensante®, Gelacril® and Polacril® polymers. BASF sells Luvigel® and Sumitomo Seika sells Aqupec®. The following companies market their corresponding thickener polymers: Goldschmidt AG—TX®; Nihon—Junlan®; Clariant—Aristoflex®; Alban Muller International—Amigel®; Corel Pharma Chem—Acrypol®; Elementis—Rheolate®; Wako Pure Chemical Ind—Hiviswako®; Rhome & Haas—Aculyn® series; Ciba Specialty Chemicals—Salcare® series; ISP—Stabileze® series; National Starch and Chemical—Structure® series; and Seppic—Capigel® series, Sepigel® series and Simulgel® series.

Other Additives

Many personal care products may benefit from the use of complexed cationic ingredients of this invention if anionic polymeric rheology modifiers or thickeners are also employed in such products. Such personal care products are intended for use in the treatment of keratinous substances such as hair, nails, skin, lips or eyelashes. More specifically, they include various hair formulations such as shampoos, rinses, gels, dyes, preparations conditioners, mousses, hot oil treatment and products for shaping or styling hair, perming or straightening preparations, setting lotions and blow-drying lotions; skin creams, lotions and sanitizers; and products that are applied on the lips, nails and eyelashes. These personal care products usually will also contain additives to provide specific desirable properties for specific product application. Such additives are exemplified below, but other additional additives may also be used as needed or desired.

Conditioning Agents:

A personal care product containing a composition of the present invention also may include from about 0.1% to about 10%, particularly about 0.5% to about 10%, and preferably from about 1.0% to about 5.0%, by weight of a non-volatile silicone compound or other conditioning agent(s), preferably a water-insoluble, emulsifiable conditioning agent. The preferred non-volatile silicone compound is a polydimethylsiloxane compound, such as a mixture, in about a 3:1 weight ratio, of a low molecular weight polydimethylsiloxane fluid and a higher molecular weight polydimethylsiloxane gum. The non-volatile polydimethylsiloxane compound is added to the composition of the present invention in an amount sufficient to provide improved combing and improved feel (softness) to the hair.

Another type of a silicone conditioning agent is “silicone gums” which are those nonfunctional siloxanes having a viscosity of from about 5 to about 600,000 centistokes at 25° C. Preferred silicone gums include linear and branched polydimethylsiloxanes. Silicone gums useful in compositions of the present invention are available from a variety of commercial sources, including General Electric Company, Dow Corning.

Also useful as conditioning agents are the so-called rigid silicones, as described in U.S. Pat. No. 4,902,499, herein incorporated by reference, having a viscosity above 600,000 centistokes at 20° C., e.g. 700,000 centistokes plus, and a weight average molecular weight of at least about 500,000 illustrated by the following formula:

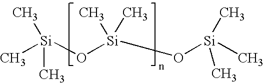

Other conditioning agents are the ‘so called’ “dimethicone copolyols” which may be linear or branched that may be block or random copolymers. Preferably, the dimethicone copolyols are block copolymers having one or more polysiloxane blocks and one or more polyether blocks, for instance ethylene oxide and propylene oxide.

Preferably, the weight ratio of ethylene oxide (C₂H₄O) to propylene oxide (C₃H₈O) in the dimethicone copolyols is from 100:0 to 35:65. The viscosity of the dimethicone copolyols as 100 percent actives at 25° C. is preferably from 100 to 4000 centistokes. The dimethicone copolyols are available from suppliers found in the International Cosmetic Ingredients Dictionary, 5th Edition, 1993, published by the CTFA in Washington D.C.

Another particularly suitable conditioning agent that can be included is a volatile hydrocarbon, such as a hydrocarbon including from about 10 to about 30 carbon atoms, that has sufficient volatility to slowly volatilize from the hair after application of the aerosol or non-aerosol styling aid composition. The volatile hydrocarbons provide essentially the same benefits as the silicone conditioning agents. The preferred volatile hydrocarbon compound is an aliphatic hydrocarbon including from about 12 to about 24 carbon atoms, and having a boiling point in the range of from about 100° C. to about 300° C. Examples of volatile hydrocarbons useful in the composition of the present invention are the commercially-available compounds PERMETHYL 99A and PERMETHYL 101A, available from Permethyl Corporation, Frazer, Pa. A volatile hydrocarbon compound is useful in the composition of the present invention either alone, in combination with another volatile hydrocarbon, or in combination with a volatile silicone. Examples of other suitable water-insoluble conditioning agents that can be incorporated into the composition of the present invention include the following: polysiloxane polyether copolymers; polysiloxane polydimethyl dimethylammonium acetate copolymers; acetylated lanolin alcohols; lauryl dimethylamine oxide; a lanolin-derived extract of sterol on sterol esters; lanolin alcohol concentrate; an isopropyl ester of lanolin fatty acids; isopropyl ester of lanolin fatty acids; oleyl alcohol; stearyl alcohol; stearamidopropyl dimethyl myristyl acetate; a polyol fatty acid; a fatty amido amine; cetyl/stearyl alcohol; tris(oligoxyethyl)alkyl ammonium phosphate; an aminofunctional silicone; lapyrium chloride; isopropyl ester of lanolic acids; ethoxylated (30) castor oil; acetylated lanolin alcohol; fatty alcohol fraction of lanolin; a mineral oil and lanolin alcohol mixture; high molecular weight esters of lanolin; quaternium-75; vinylpyrrolidone/ dimethyl amino-ethylmethacrylate copolymer; 5 mole ethylene oxide adduct of soya sterol; 10 mole ethylene oxide adduct of soya sterol; stearic acid ester of ethoxylated (20 mole) methyl glucoside; sodium salt of poly-hydroxycarboxylic acid; hydroxylated lanolin; isostearamidopropyl dimethylamine lactate; isostearamidopropyl morpholine lactate; oleamidopropyl dimethylamine lactate; linoleamidopropyl dimethylamine lactate; stearamidopropyl dimethylamine lactate, ethylene glycol monostearate and propylene glycol mixture; stearamidopropyl dimethylamine lactate; cetearyl alcohol mixture; cetearyl alcohol; tallow imidazolinum methosulfate; stearyl trimonium methosulfate; mixed ethoxylated and propoxylated long chain alcohols; stearamidopropyl dimethylamine lactate; polonitomine oxide; oleamine oxide; stearamine oxide; soya ethyldimonium ethosulfate; ricinolamidopropyl ethyldimonium ethosulfate; N-(3-isostearamido-propyl)-N,N-dimethyl amino glycolate; N-(3-isostearamidopropyl)-N,N dimethyl amino gluconate; hydrolyzed animal keratin; ethyl hydrolyzed animal keratin; avocado oil; sweet almond oil, grape seed oil; jojoba oil; apricot kernel oil; sesame oil; hybrid safflower oil; wheat germ oil; cocamidoamine lactate; ricinoleamido amine lactate; stearamido amine lactate; stearamido morpholine lactate; isostearamido amine lactate; isostearamido morpholine lactate; wheat germamido dimethylamine lactate; behenamidopropyl betaine; ricinoleamidopropyl betaine; wheat germamidopropyl dimethylamine oxide; disodium isostearaimido MEA sulfosuccinate; disodium oleamide PEG-2 sulfosuccinate; disodium oleamide MEA sulfosuccinate; disodium ricinoleyl MEA sulfosuccinate; disodium wheat germamido MEA sulfosuccinate; disodium wheat germamido PEG-2 sulfosuccinate; polyethylene glycol (400) mono and distearates; synthetic calcium silicate; isostearic alkanolamide; ethyl esters of hydrolyzed animal protein; blend of cetyl and stearyl alcohols with ethoxylated cetyl or stearyl alcohols; amido amines; polyamido amines; palmityl amido betaine; propoxylated (1-20 moles) lanolin alcohols; isostearamide DEA; and hydrolyzed collagen protein. When one or more of these water-insoluble conditioning agents is included in the composition of the present invention in an amount of about 0.5% to about 10% by total weight of the composition, the composition also can include a suspending agent for the conditioning agent, in an amount of about 0.5% to about 10%, by total weight of the composition. The particular suspending agent is not critical and can be selected from any materials known to suspend water-insoluble liquids in water. Suitable suspending agents are for example, distearyl phthalamic acid; fatty acid alkanolamides; esters of polyols and sugars; polyethylene glycols; the ethoxylated or propoxylated alkylphenols; ethoxylated or propoxylated fatty alcohols; and the condensation products of ethylene oxide with long chain amides. These suspending agents, as well as numerous others not cited herein, are well known in the art and are fully described in the literature, such as McCutcheon's Detergents and Emulsifiers, 1989 Annual, published by McCutcheon Division, MC Publishing Co. A nonionic alkanolamide also is optionally included in an amount of about 0.1% to about 5% by weight in the styling aid compositions that include a conditioning agent to provide exceptionally stable emulsification of water-insoluble conditioning agents and to aid in thickening and foam stability. Other useful suspending and thickening agents can be used instead of the alkanolamides such as sodium alginate; guar gum; xanthan gum; gum arabic; cellulose derivatives, such as methylcellulose, hydroxybutylcellulose, hydroxyethylcellulose, hydroxypropylcellulose and carboxymethylcellulose; and various synthetic polymeric thickeners, such as the polyacrylic acid derivatives. Suitable alkanolamides include, but are not limited to, those known in the art of hair care formulations, such as cocamide monoethanolamide (MEA), cocamide diethanolamide (DEA), soyamide DEA, lauramide DEA, oleamide monoisopropylamide (MIPA), stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA and combinations thereof.

Neutralizinp Agents:

In formulations containing anionic rheology modifiers, it is often necessary to neutralize the polymeric thickener. Neutralization is accomplished with one or more inorganic bases such as sodium hydroxide, potassium hydroxide, ammonium hydroxide and/or ammonium carbonate. Useful neutralizing organic bases are primary, secondary and tertiary amines and the water soluble alkanol amines such as monoethanolamine (MEA), diethanolamine (DEA), triethanolamine (TEA), 2-methyl-2-amino-1-propanol (AMP), 2-amino-2-methyl-propanol and 2-amino-2-methyl-1,3-propanediol, respectively, 2-dimethylaminoethanol N,N-dimethyl-ethanolamine), 3-dimethylamino-1-propanol, 3-dimethylamino-2-propanol, 1-amino-2-propanol, and the like, monoamino glycols, and the like, which help solubilize the polymer in water solutions. The level of neutralization required varies for each polymer. The block copolymers become soluble in water and hydroalcoholic solutions at 20% to 100% neutralization, and at all described levels of water/alcohol/ propellant solutions. The pH of these solutions usually ranges from 4 to 12 but generally will be between 5 and 8. The lowest neutralization level needed to render the polymer water soluble or dispersible depends on the composition of the block polymer, and the amount of alcohol, water, and propellant.

Aerosol Propellant Gas:

The propellant gas included in aerosol compositions can be any liquefiable gas conventionally used for aerosol containers. Examples of materials that are suitable for use as propellants are trichlorofluoromethane, dichlorodifluoromethane, dichlorotetrafluoroethane, monochlorodifluoro-methane, trichlorotrifluoroethane, dimethyl ether, propane, n-butane and isobutane, used singly or admixed. Water-soluble gases such as dimethyl ether, carbon dioxide, and/or nitrous oxide also can be used to obtain aerosols having reduced flammability. Water-immiscible, liquified, hydrocarbon and halogenated hydrocarbon gases such as propane, butane and chlorofluorocarbons can be used advantageously to deliver the contents of the aerosol container without the dramatic pressure drops associated with other immiscible gases. Here there is no concern for the head space to be left inside the aerosol container, because the liquified gas will sit on top of the aqueous formulation and the pressure inside the container is always the vapor pressure of saturated hydrocarbon vapor. Other insoluble, compressed gases such as nitrogen, helium and fully-flourinated oxetanes and oxepanes also are useful to deliver the compositions from aerosol containers. Other means of delivery of the above-described aqueous styling aid compositions include, pump sprayers, all forms of bag-in-can devices, in situ carbon dioxide (CO.sub.2) generator systems, compressors, and the like. The amount of the propellant gas is governed by normal factors well known in the aerosol art. For mousses, the level of propellant is generally from about 3% to about 30%, preferably from about 5% to about 15% of the total composition. If a propellant such as dimethyl ether utilizes a vapor pressure suppressant (e.g., trichlorethane or dichloromethane), for weight percentage calculations, the amount of suppressant is included as part of the propellant.

The final products may optionally contain one or more fixative resins. Examples of hair fixative resins include synthetic polymers such as polyacrylates, polyvinyls, polyesters, polyurethanes, polyamides and mixtures thereof; polymers derived from natural sources such as modified cellulose, starch, guar, xantham, carragenan and blends thereof. These resins may have cationic, anionic, nonionic, ampholytic or zwitterionic in character. They may be soluble, dispersible or insoluble in water and hydroalcoholic formulations glass transition temperature, Tg, may be in the range from −50° C. to 200° C.

Another class of organosilicones that may be advantageously incorporated in hair styling compositions are silicone resins which are non-polar silsesquioxanes. These resins are film forming and aid in imparting good cure retention property to the composition. The silsesquioxanes have a formula selected from the group consisting of

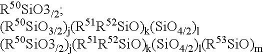

and hydroxy, alkoxy, aryloxy, and alkenoxy, derivatives thereof, wherein R⁵⁰, R⁵¹, R⁵² and R⁵³, are selected from the group consisting of alkyl, alkenyl, aryl, and alkylaryl, radicals having from one to twenty carbon atoms; and j, k, l, and m, are each integers having a value of from zero to about one thousand, with the proviso that the sum of integers j and l must be at least one.

The nonpolar silsesquioxane silicone resin materials conforming to any one of the above-specified generic formulas are commercially available from the Dow Corning Corporation, Midland, Mich.

These nonpolar silsesquioxanes can be incorporated into hair styling formulations containing the block copolymers of the invention provided a solvent, such as ethanol or any other appropriate solvent is present in the formulation, either above or in a mixture with water.

The organosilicone compound is present in the mixture at a level from about 0.1 to about fifty percent by weight based on the weight of the mixture. Preferably, the organosili-cone compound is present in the mixture at a level from about three to about thirty percent by weight based on the weight of the mixture. The solvent may be water, a hydrocarbon, an alcohol, or a blend of alcohol and water. Other solvents which may be employed include supercritical fluids such as supercritical carbon dioxide and nitrogen; volatile silicones including linear and cyclic siloxanes; non-volatile hydrocarbons; and in some instances, aqueous emulsion systems may also be appropriate. Where the solvent is hydrocarbon, it is preferred to employ materials such as dimethylether, liquefied petroleum gas, propane, and isobutane. In the event the solvent is an alcohol, some appropriate materials are methanol, ethanol, and isopropanol.

One example of a silsesquioxane may be represented by the formula

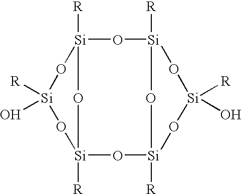

Another additive that may be incorporated is a soluble surface tension reducing compound. It is any soluble compound which reduces the surface tension between the hair styling composition and the gaseous atmosphere above the hair styling composition. By “gaseous atmosphere” we mean a propellant or air. The soluble surface tension reducing compound may be for example a plasticizer or surfactant in a hair styling composition. The soluble surface tension reducing compound includes for example dimethiconecopolyols, panthenol, fluorosurfactants, glycerin POE, PPG 28 Buteth 35, PEG 75 lanolin, oxtoxynol-9, PEG-25 hydrogenated castor oil, polyethylene glycol 25 glyceryl trioleate, oleth-3 phosphate, PPG-5-ceteth-10 phosphate, PEG-20 methyl glucose ether, or glycereth-7-triacetate, glycereth-7-benzoate or combinations thereof. Preferably the soluble surface tension compound is dimethiconecopolyols, panthenol, glycereth-7-benzoate, or combinations thereof.

The soluble surface tension reducing compound is typically present in the low beading, low VOC hair styling composition at a concentration of from 0.01 to 1 weight percent, and more preferably at a concentration of from 0.01 to 0.25 weight percent, based on the total weight of the composition.

Also useful additives are plasticizing compounds. The first class of plasticizing compounds are soluble polycarboxylic acid esters. The polycarboxylic acid esters have a carbon backbone of from 3 to 12 carbon atoms and 3 or more C₁ to C₅ alkyl carboxylate groups attached thereto. Suitable polycarboxylic acid esters include, for example, triethyl citrate, tributyl citrate, triethyl phthalate, tributyl phthalate, tripentyl phthalate or combinations thereof. Preferably, the polycarboxylic add esters are selected from triethyl citrate, tributyl citrate, tributyl phthalate, or combinations thereof and more preferably are selected from triethyl citrate, tributyl citrate, or combinations thereof. The plasticizing compounds are preferably added to a hair styling composition to provide a total concentration of from 0.01 to 1.0 weight percent plasticizing compounds, more preferably 0.1 to 0.5 weight percent plasticizing compounds, based on the total weight of a hair styling composition.

The formulation may optionally contain one or more nonactive adjuvants in an amount up to about 5 wt. % based on the total composition. Such nonactive additives include a corrosion inhibitor, a surfactant, a film hardening agent, a hair curling agent, a coloring agent, a lustrant, a sequestering agent, a preservative and the like. Typical corrosion inhibitors include methylethyl amine borate, methylisopropyl amine borate, inorganic hydroxides such as ammonium, sodium and potassium hydroxides, nitromethane, dimethyl oxazolidine, 2-dimethylamino-2-methyl-1-propanol, and aminomethyl propanol.

Emollients like Guerbet alcohols and esters thereof, silicone derivatives, beeswax, C12-15 alcohols, benzoate, mineral oil, capric triglycerides, cetearyl alcohol, ceteareth-20, castol oil, isohexadecane, isopropyl myristate, isopropyl palmitate, cetearyl octanoate and petrolatum;

UV-absorbers like butyloctyl salicylate, octylmethoxycinnamate, avobenzone, benzophenone-3 and benzophenone-4, octyl salicylate, para-aminobenzoric acid (PABA), octyldimethyl PABA, hindered cyclic amine UV-light stabilizers based on 3.5-hindered piperidines available as TINUVIN® series of products from Ciba Specialty Chemicals or 3.5-hindered-2-keto-piperazinones.

Surfactants like alcohols, alcohol ethoxylates, alkanolamine-derived amides, ethoxylated amides, amine oxides, ethoxylated carboxylic acids, ethoxylated glycerides, glycol esters and derivatives thereof, monoglycerides, polyglyceryl esters, polyhydric alcohol esters and ethers, sorbitan/sorbitol esters, trimesters of phosphoric acid, ethoxylated lanolin, silicone polyethers, PPO/PEO ethers, alkylpolyglycosides, acyl/dialkyl ethylenediamines and derivatives, n-alkyl amino acids, acyl glutamates, acyl peptides, sarcosinates, taurates, alkanoic acids, carboxylic acid esters, carboxylic acid ethers, phosphoric acid esters and salts, acyl isethionates, alkylaryl sulfonates, alkyl sulfonates, sulfosuccinates, alkyl ether sulfates and alkyl sulfates.

Carrier Vehicle:

Polar solvents are typically used to prepare the cosmetic or hair compositions. Water, glycols and alcohols are preferably used. The optional alcohol employed in the composition is an aliphatic straight or branched chain monohydric alcohol having 2 to 4 carbon atoms. Isopropanol and especially ethanol are preferred. The concentration of the alcohol in the composition should be less than about 40% by weight, and surprisingly can be as low as 0%, preferably 0-30% by weight and more preferably 5-20% by weight. Some alcohol, in an amount of about 2% to about 10% by weight.

A non-aerosol, low VOC, pump hair spray composition is provided herein which is capable of being applied by the user as a fine spray mist, which dries rapidly on the hair, and which provides low curl droop and effective curl retention properties thereon. The composition consists essentially of a copolymer as a hair fixative polymer, and a mixture of alcohol, water and dimethoxymethane (DMM) as cosolvents therefor. Such formulations may be prepared as anhydrous formulas as well as all water systems, and both as hair sprays or as mousse products. For these applications, it is preferable to use lower molecular weight hair fixative copolymers and the sprayed droplets size should be as small as practical to achieve fast drying of the film. Preferably, the hair fixative polymer is present at a solids level of about 1-15%, the alcohol in an amount of about 50-70%, water at 10-30%, and DMM at 10-30%, by weight of the composition.

### COMPATIBILITY OF COMPLEXED CATIONICS WITH CARBOPOL® POLYMERS

Experimental

A 0.5% Carbopol® polymer mucilage was prepared and neutralized to pH 7.0-7.5 with sodium hydroxide (PART A). Separately, a solution containing the appropriate levels of cationic material, silicone and neutralizing agent (sodium hydroxide or citric acid, to pH 7.0-7.5) was prepared (PART B). Twenty parts PART B was added to eighty parts PART A. Viscosity was measured on a Brookfield RV Viscometer at 23° C. and 20 rpm. Turbidity was measured on a Micro 1000 Turbidimeter.

Results and Discussion

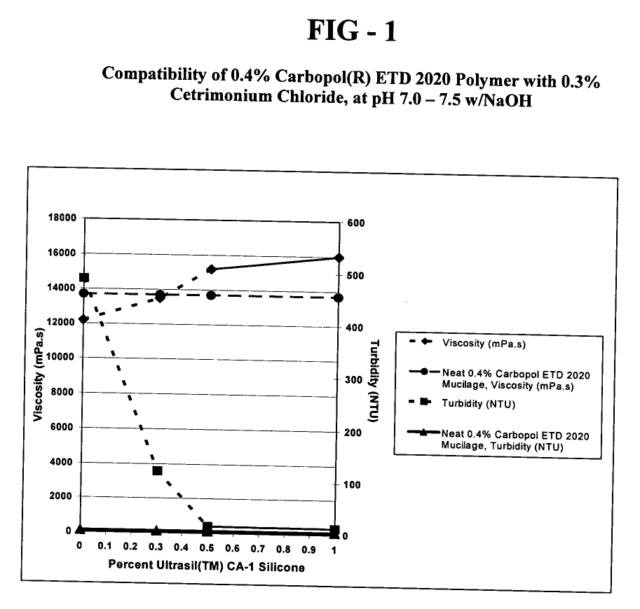

FIGS. 1-3 show low molecular weight quaternary ammonium compounds (cetrimonium chloride, stearalkonium chloride and olealkonium chloride) complexed with Ultrasil™ CA-1 silicone (dimethicone copolyol phthalate or DMC phthalate), and added to a Carbopol® ETD 2020 polymer mucilage. As the level of Ultrasil™ CA-1 silicone was increased, the viscosity increased and turbidity trended toward that of a gel containing no cationic material whatsoever. When enough anionic silicone was used, precipitate was no longer generated. A dotted line in the Figures signifies presence of precipitate. Conversely, a solid line signifies absence of precipitate.

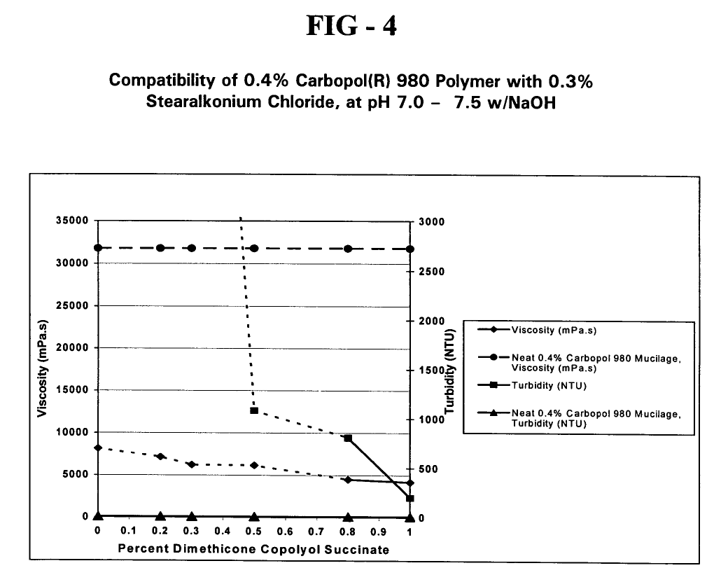

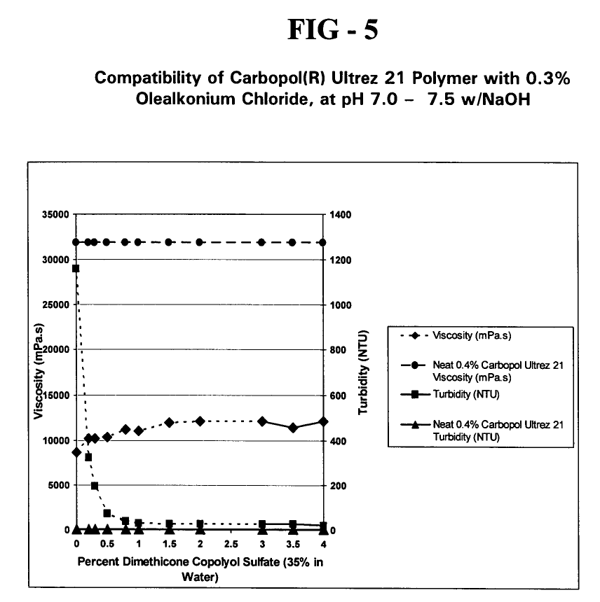

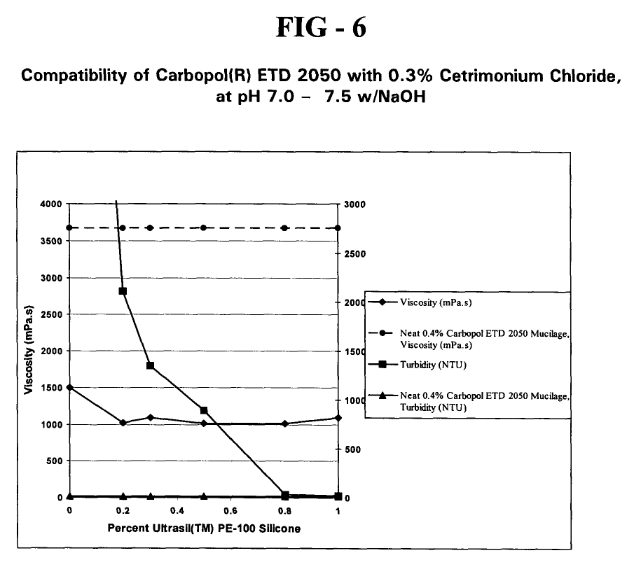

This concept is broad in scope and applies to a wide range of Carbopol® polymers and anionic silicones, evidenced by FIGS. 4-6. FIG. 4 shows the results of a DMC succinate-stearalkonium chloride complex in a mucilage made with Carbopol® 980 polymer. FIG. 5 shows the results of a DMC sulfate-olealkonium chloride complex in a mucilage made with Carbopol® Ultrez-21 polymer. FIG. 6 shows the results of a DMC phosphate-cetrimonium chloride complex in a mucilage made with Carbopol® ETD 2050 polymer (acrylates/C10-30 alkyl acrylated crosspolymer). Viscosity was not recovered in all cases, but turbidity reduction was common to all of the examples. Precipitation elimination was common to all of examples except for the system depicted in FIG. 6, which showed no precipitation at any of the tested conditions. Some systems (FIG. 5) required more silicone to achieve the desired effect.

Cationic ingredients are typically used at lower pH values (4-6). Gels with minimum ingredients, such as those studied in FIGS. 1-6, are very sensitive at low pH values and produce curves that are too noisy to clearly show trends. While FIGS. 1-6 are valuable for academic purposes, more practical demonstrations of the ability of anionic silicones to compatibilize cationics and Carbopole polymers are shown below in FORMULATIONS.

Additional testing (Table 1) shows that the dimethicone copolyol precursor to Ultrasil™ CA-1 silicone has very limited capacity to compatibilize the cationic ingredient and the thickener when compared to CA-1. This demonstrates that the observed compatibilization is due to complexation, and not steric effects of the silicone.

TABLE 1
Compatibility of Low MW Quats in a Gel Containing 0.4%
Carbopol ® ETD 2020 Polymer at pH 7.0-7.5

| | | Cetri- | Stearal- | | | |
|---|---|---|---|---|---|---|
| Ultrasil ™ | Dimeth- | monium | konium | | | |
| CA-1 | icone | Chlo- | Chlo- | Viscos- | | |
| Silicone, | Copolyol | ride | ride | ity | Turbidity, | Precip- |
| % | % | % | % | mPa · s | NTU | itate |
| — | — | 0.30 | — | 12,200 | 487 | Yes |
| — | 0.50 | 0.30 | — | 2,650 | 459 | Yes |
| 0.50 | — | 0.30 | — | 15,200 | 13.1 | No |
| — | — | — | 0.30 | 10,500 | >10,000 | Yes |
| — | 0.50 | — | 0.30 | 9,150 | 164 | Yes |
| 0.50 | — | — | 0.30 | 11,750 | 22.1 | No |

NOTE:
A neutral gel containing only 0.4% Carbopol ® ETD 2020 polymer and sodium hydroxide was measured to have a viscosity of 13,700 mPa · s and a turbidity of 3.8 NTU.

Enhanced compatibility with Carbopol® polymers does not appear to be limited to low MW quaternary ammonium compounds. Polyquaternium compounds and divalent cations were screened, with positive results (Table 2).

TABLE 2
Compatibility of Polyquaternium Compounds and Divalent
Cations in a Gel Containing 0.4% Carbopol ® ETD 2020
Polymer at pH 7.0-7.5

| | | Active | | | |
|---|---|---|---|---|---|
| Ultrasil ™ | | Cationic | Viscos- | Tur- | |
| CA-1 | Cationic | Material, | ity | bidity, | Precip- |
| Silicone, | Material | % | mPa · s | NTU | itate |
| — | Polyquaternium-7* | 0.30 | 11,000 | 11.2 | Yes |
| 0.50 | Polyquaternium-7* | 0.30 | 8,100 | 10.3 | No |
| — | Polyquaternium-11⁺ | 0.30 | 16,100 | 7.7 | Yes |
| 0.50 | Polyquaternium-11⁺ | 0.30 | 12,650 | 7.4 | No |
| — | Calcium Acetate | 0.10 | 7,400 | 6.7 | Yes |
| | Monohydrate | | | | |
| | (Fisher) | | | | |
| 0.15 | Calcium Acetate | 0.10 | 5,700 | 4.7 | No |
| | Monohydrate | | | | |
| | (Fisher) | | | | |

NOTE:
A neutral gel containing only 0.4% Carbopol ® ETD 2020 and sodium hydroxide was measured to have a viscosity of 10,200 mPa · s and a turbidity of 4.1 NTU.
*Merguat 550 from Nalco ⁺ Gafguat 734 from ISP

Without the inclusion of the Ultrasil™ CA-1 silicone, the addition of polyquaternium compound or multivalent cation resulted in precipitation. When CA-1 was included in the preparation of the sample, no precipitate was present, and the viscosity readings were reduced. This reduction in viscosity is an indication of reduced “ionic crosslinking” of Carbopol® polymer by the tested cationic materials. Turbidity was already very low at the tested conditions and seemed to be unaffected by CA-1 inclusion.

In general, the best results were observed when the silicone and the cationic material were blended in aqueous media, adjusted to match the pH of gel, and finally added to the gel. The appropriate silicone-to-cationic ratio was found to be formulation dependent. It increases as the usage level of the cationic is raised, and as the pH decreases.

Conclusions

Low molecular weight quaternary ammonium compounds, when complexed with anionic silicones, can be made compatible with systems containing Carbopol® polymer. Increased compatibility is defined as reduced tendency to form precipitation, reduced turbidity, and/or improvement in viscosity profile. Anionic silicones also compatibilize Carbopol® polymers with polyquaternium compounds and divalent cations.

### EFFICACY

The following tests show that complexation of the cationic material does not interfere with the ability of the cationic material to deposit on anionic hair. Such interference would negatively affect conditioning properties such as wet comb-through, for which low MW quats are most commonly used to improve.

The Rubine dye test is commonly used to measure deposition of cationic ingredients on hair. It involves soaking pre-conditioned yak hair in a solution of anionic red dye. Yak hair is used because of its availability and lack of color. The hair's uptake of red dye is related to the amount of cationic material already deposited. A calorimeter measures hair color according to the CIE-LAB ternary coordinate system. Positions on the three dimensionless axes (L*=lightness, a*=red-green and b*=yellow-blue), which correspond to color differences perceived by human vision, are assigned based on the reflectance specrum of the hair sample. The a* axis is used to guage the uptake of red dye.

Wet comb-through is the total work required to pull the wet hair completely through a comb five times, as measured by a tensiometer.

Experimental

Rubine Dye Test

Clipped yak belly hair tresses (about 3 g, 18 cm each) from International Hair Importers, Inc. were washed with a 10% solution of sodium lauryl sulfate. Background color scans were taken using a Hunter LabScan II Colorimeter with Universal Software V.2.10. The tresses were dampened with DI water, soaked in a solution of conditioner for a total of three minutes and rinsed for one minute with lukewarm tap water. Excess water was wrung out. The tresses were soaked in a solution of 0.5% pyrazol dye (pH 3.5 with acetic acid) for five minutes, and again rinsed for one minute using lukewarm tap water. The tresses were allowed to dry at room temperature. Color measurements were repeated. Tests were performed in duplicate.

Wet Comb-Through Test

Bleached Virgin European brown clipped human hair tresses (about 3 g, 18 cm each) from International Hair Importers, Inc. were washed with a 10% solution of sodium lauryl sulfate. The tresses were dampened with DI water, soaked in a solution of conditioner for a total of three minutes, and rinsed for one minute with lukewarm tap water. Each wet tress was placed in an A/TG tensile grip of a TA-XT21 Texture Analyser (Texture Technology Corp.) at 23° C. and 50% relative humidity. The tensile grip was lowered so that the hair rested in the designated section of the exposed fine tines of the comb (model 220041 from Sally's Beauty Supply). The tress was raised at a rate of 3.0 mm/s until it had completely passed through the comb. The force needed to raise the tress was recorded as a function of distance. This was repeated four times, for a total of five pulls. The areas under the force vs. distance curves were calculated and summed, yielding total work performed. Tests were performed in triplicate.

Results and Discussion

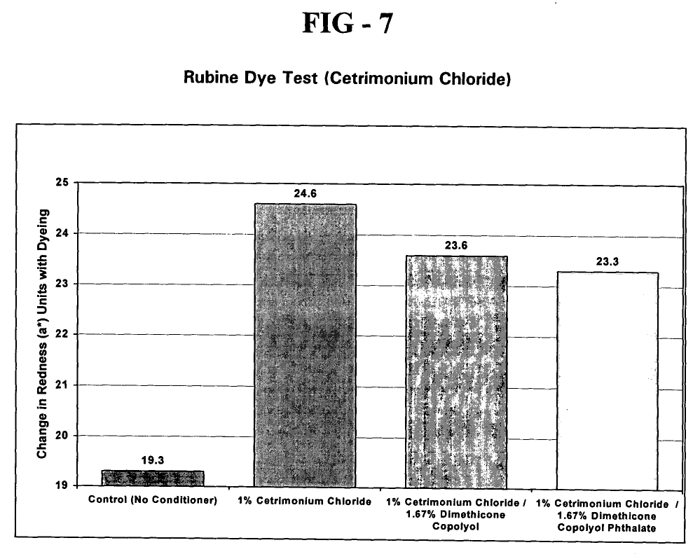

FIGS. 7-9 show the results of Rubine dye tests.

FIG. 7 shows the hair treated with a conditioning system comprising cetrimonium chloride complexed with Ultrasil™ CA-1 silicone. A conditioning system comprising the same quaternary compound blended with the dimethicone copolyol precursor to CA-1 was also tested (this is the exact same conditioning system, without the ability to complex). No significant deposition difference was observed between the two conditioning systems, which suggests complexation does not affect cationic deposition on hair. A conditioning system consisting only of cetrimonium chloride deposited slightly better than the other two, which can be attributed to the absence of steric effects from other ingredients.

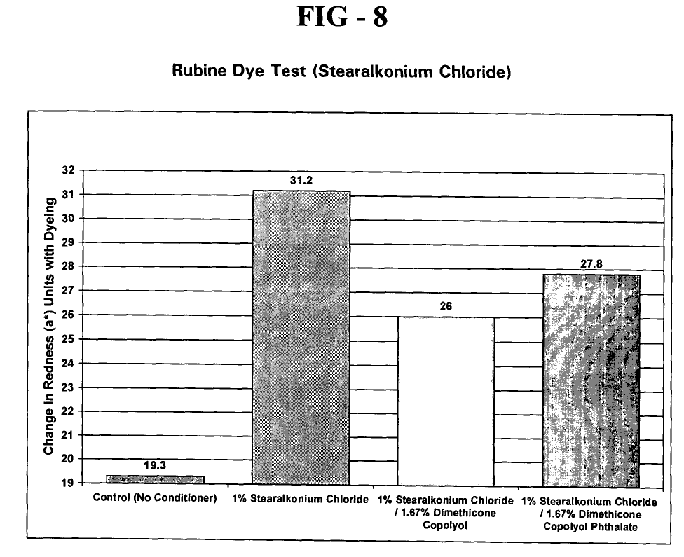

FIG. 8 shows similar results with stearalkonium chloride. Again, complexation is shown to not reduce deposition. In fact, more deposition was measured with the silicone-complexed conditioning system than with the silicone-blended conditioning system.

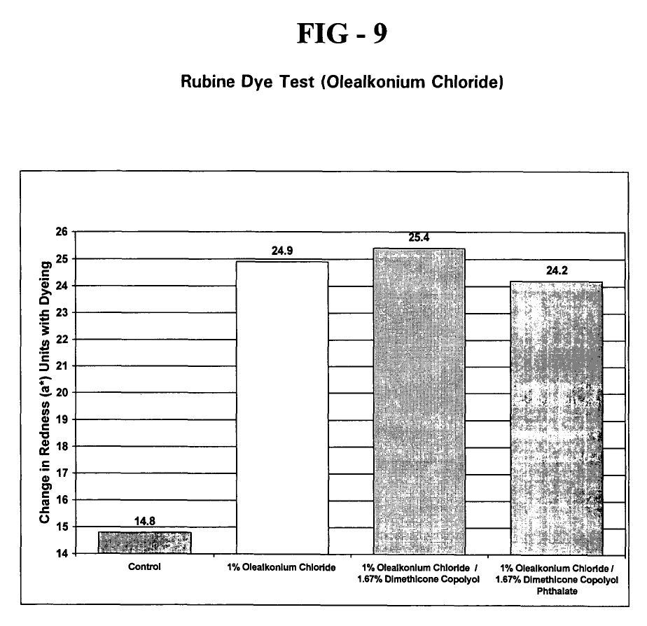

FIG. 9 shows no significant differences between three olealkonium chloride conditioning systems. Although differences were measured, they were of roughly the same magnitude as the differences between duplicate tresses for a given conditioning system.

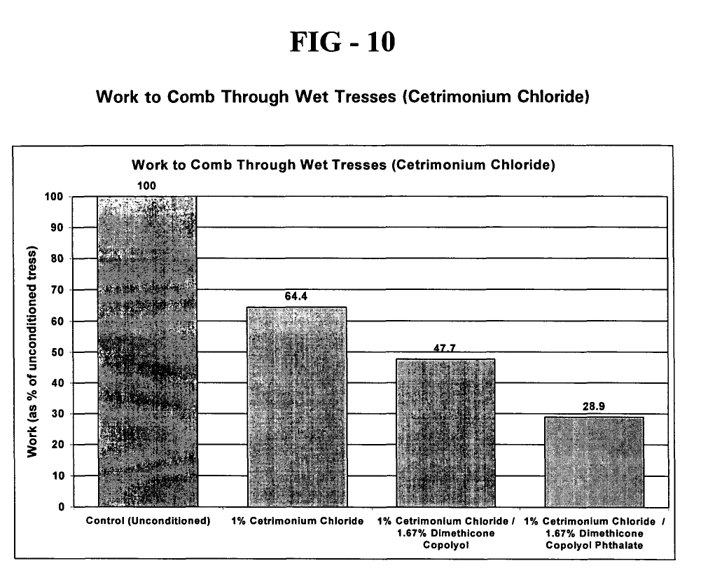

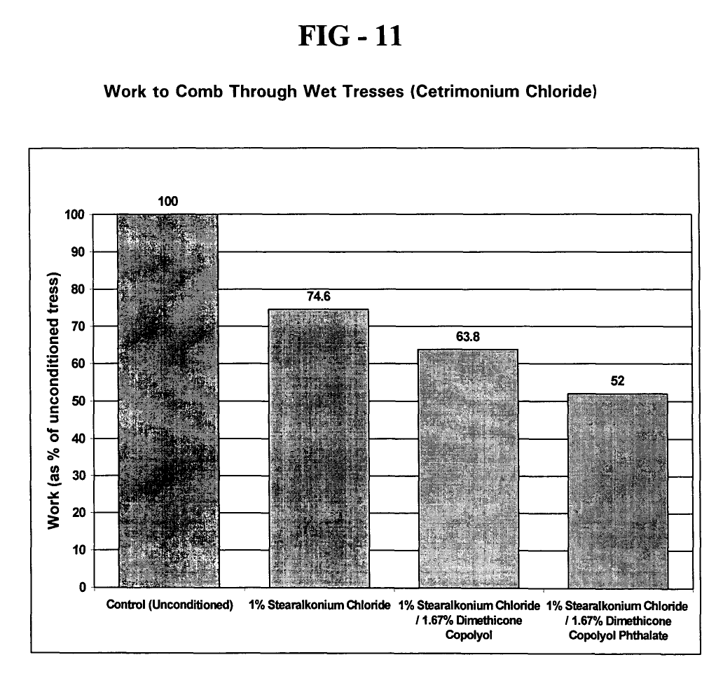

Two conditioning systems (cetrimonium chloride complexed with dimethicone copolyol phthalate, Ultrasil CA-1, which is anionic, and cetrimonium chloride blended with dimethicone copolyol, the precursor to CA-1, which is not anionic) whose cationic components deposit equally on hair (as shown by the Rubine dye tests) were shown to perform differently in wet comb-through tests (FIG. 10). The silicone complex had better comb-through than the silicone blend, both of which had better comb-through than a simple cetrimonium chloride solution. The differences can be attributed to differing levels of silicone on the hair. It can be concluded that not only does cationic material that has been complexed still deposit on the hair, but it brings the anionic silicone along with it, in quantities greater than the anionic silicone would otherwise deposit (assuming anionic silicone and dimethicone copolyol have similar deposition on hair). This conclusion is supported by FIG. 11, which shows the same experiment run on stearalkonium chloride. The stearalkonium tests showed much greater tress-to-tress variation, but the overall conditioning system rankings were the same as with cetrimonium chloride.

Conclusions

Complexing low MW quaternary ammonium compounds with anionic silicone does not reduce the deposition of the quat onto hair, but appears to increase the deposition of silicone onto hair which enhances conditioning properties.

### FORMULATIONS

Clear Conditioning Styling Gel

This crystal clear formula contains cetrimonium chloride, Ultrasil™ CA-1 silicone and Carbopol® ETD 2020 polymer. It demonstrates the utility of complexation at realistic pH levels.

| | Weight | | Trade Name |
|---|---|---|---|
| Ingredient | Percent | Function | (Supplier) |
| Part A | | | |
| Deionized Water | QS | Diluent | |
| Acrylates/ | 0.55 | Rheology | Carbopol ® ETD 2020 |
| C10-C30 Alkyl | | Modifier | Polymer (Noveon) |
| Acrylates | | | |
| Crosspolymer | | | |
| Sodium Hydrox- | 0.20 | Neutralizing | |
| ide (10%) | | Agent | |
| Part B | | | |
| Deionized Water | 12.0 | Diluent | |
| VP/VA Copoly- | 7.50 | Fixative | Luviskol ® VA 73W |
| mer | | | (BASF) |
| Sodium Hydrox- | 0.20 | Neutralizing | |
| ide (1%) | | Agent | |
| Part C | | | |
| Deionized Water | 2.50 | Diluent | |
| Benzophenone-4 | 0.05 | UV Ab- | Uvinul ® MS-40 |
| | | sorber | (BASF) |
| Part D | | | |
| Deionized Water | 2.50 | Diluent | |
| Disodium EDTA | 0.05 | Chelating | Versene NA (Dow) |
| | | Agent | |
| Part E | | | |
| Deionized Water | 12.0 | Diluent | |
| Dimethicone | 0.10 | Conditioner/ | Ultrasil ™ CA-1 |
| PEG-7 Phthalate | | Compat- | Silicone (Noveon) |
| | | ibilizing | |
| | | Agent | |
| Cetrimonium | 0.20 | Conditioner | Genamin CTAC |
| Chloride (30%) | | | (Clariant) |
| Part F | | | |
| DMDM Hydan- | 0.30 | Preservative | Glydant ® (Lonza) |
| toin | | | |
| Sodium Hydrox- | QS to | Neutralizing | |
| ide (10%) | pH 5.0-5.3 | Agent | |

Procedure:
Part A was prepared - Carbopol ® ETD 2020 polymer was sifted into
water and neutralized. Part B was prepared and added to Part A. Part C
was prepared and added. Part D was prepared using heat and added. Part E
was prepared and added. The ingredients of Part F were added separately.
Properties of Styline Gel

| | |
|---|---|
| pH | 5.3 |
| Viscosity @ 20 rpm, 20 C. (mPa · s) | 16,400 |
| Turbidity on Micro 1000 Turbidimeter | 8 |
| (NTU) | |
| Stability | Passed 3 months accelerated, |
| | 45° C. |
| Freeze-Thaw Stability (3 cycles) | Pass |

When preparation of the preceding formulation was repeated without Ultrasil™ CA-1 silicone, precipitate formed immediately upon addition of the cetrimonium chloride.

Clear Rinse-Off Conditioning Gel

This unique rinse-off formula contains cetrimonium chloride, dimethicone copolyol sulfate and Carbopol® ETD 2020 polymer. It shows how complexation can be used to make formulations that would otherwise not be possible.

| Ingredient | Weight % | Function | Trade Name (Supplier) |
|---|---|---|---|
| Part A | | | |
| Deionized Water | QS | Diluent | |
| Acrylates/ | 0.50 | Rheology | Carbopol ® ETD 2020 |
| C10-C30 Alkyl | | Modifier | Polymer (Noveon) |
| Acrylates | | | |
| Crosspolymer | | | |
| Sodium Hydrox- | 0.55 (QS to | Neutralizing | |
| ide (18%) | pH 5.0-5.3) | Agent | |
| Part B | | | |
| Cetrimonium | 0.50 | Conditioner | Genamin CTAC |
| Chloride (30%) | | | (Clariant) |
| Dimethicone | 1.50 | Conditioner/ | Ultrasil ™ SA-1 Silicon |
| PEG-7 Sulfate | | Compat- | (Noveon) |
| (35%) | | ibilizing | |
| | | Agent | |
| Deionized Water | 2.50 | Diluent | |
| Citric Acid (50%) | 0.10 (QS to | Neutralizing | |
| | pH 5.0-53) | Agent | |
| Part C | | | |
| Benzophenone-4 | 0.05 | UV Ab- | Uvinul MS-40 (BASF) |
| | | sorber | |
| Silicone | 2.00 | Conditioner | Ultrasil ™ Q-8 Silicone |
| Quaternium-8 | | | |
| DMDM Hydan- | 0.30 | Preservative | Glydant ® (Lonza) |
| toin | | | |
| FD&C Yellow #5 | 0.07 | Dye | (Noveon Hilton Davis) |
| (0.1%) | | | |
| FD&C Blue #1 | 0.07 | Dye | (Noveon Hilton Davis) |
| (0.1%) | | | |
| Part D | | | |
| Fragrance | 0.20 | Fragrance | Country Apple 354-06 |
| | | | (Drom) |
| Polysorbate 20 | 0.20 | Solubilizing | Tween 20 (Uniqema) |
| | | Agent | |

Procedure:
Part A was prepared - Carbopol ® ETD 2020 polymer was sifted into
water and neutralized. Part B was blended and added to Part A. Part C
ingredients were added one at a time. Part D was blended and added.

| | |
|---|---|
| Properties of Conditioning Gel | |
| pH | 5.2 |
| Viscosity @ 20 rpm, 20 C. (mPa · s) | 12,400 |
| Turbidity on Micro 1000 Turbidimeter | 20 |
| (NTU) | |
| Stability | Passed 3 months accelerated, |
| | 45° C. |
| Freeze-Thaw Stability (3 cycles) | Pass |

When preparation of the preceding formulation was repeated without dimethicone copolyol sulfate, the final product form contained precipitate.

Clear Conditioning Styling Gel

This clear styling gel formula contains Polyquaternium-4, dimethicone copolyol succinate and Carbopol® Ultrez 21 polymer. It demonstrates the utility of the complexation in formulations containing polyquaternium compounds.

| | Weight | | Trade Name |
|---|---|---|---|
| Ingredient | Percent | Function | (Supplier) |
| Part A | | | |
| Deionized Water | 67.5 | Diluent | |
| Acrylates/ | 0.30 | Rheology | Carbopol ® Ultrez 21 |
| C10-C30 Alkyl | | Modifier | Polymer (Noveon) |
| Acrylates Cross- | | | |
| polymer | | | |
| DMDM Hydantoin | 0.30 | Preservative | Glydant ® (Lonza) |
| Aminomethyl | 0.25 (QS to | Neutralizing | AMP-95 (Angus) |
| Propanol | pH 6.8-7.0) | Agent | |
| Part B | | | |
| Deionized Water | 29.0 | Diluent | |
| Polyquaternium-4 | 1.00 | Fixative | Celquat ® H-100 |
| | | | (National Starch) |
| Dimethicone | 1.00 | Conditioner/ | Ultrasil ™ CA-2 |
| PEG-7 Succinate | | Compat- | Silicone |
| | | ibilizing | |
| | | Agent | |
| Aminomethyl | 0.25 (QS to | Neutralizing | AMP-95 ® (Angus) |
| Propanol | pH 6.8-7.0) | Agent | |

Procedure:
Part A was prepared - Carbopol ® Ultrez 21 polymer was added into
water and allowed to wet. Glydant was added. Neutralizer was added.
Part B was prepared - Polyquaternium-4 was sifted into water and mixed
until uniform. CA-2 was added, and Part B was neutralized. Part B was
added to Part A.
Properties of Styline Gel

| | |
|---|---|
| pH | 6.9 |
| Viscosity @ 20 rpm, 20 C. (mPa · s) | 17,550 |
| Turbidity on Micro 1000 Turbidimeter | 6.9 |
| (NTU) | |
| Stability | Passed 3 months accelerated, |
| | 45° C. |

When preparation of the preceding formulation was repeated without dimethicone copolyol succinate, the final product viscosity was higher (40,000 mPa.s), but the turbidity (14.5 NTU) was not optimal.

Aloe Gel

This skin moisturizer formula contains aloe extract, dimethicone copolyol succinate and Carbopol® Ultrez 21 polymer. It demonstrates the utility of the complexation in formulations containing high levels of salts.

| | Weight | | |
|---|---|---|---|
| Ingredient | Percent | Function | Trade Name (Supplier) |
| Part A | | | |
| Deionized Water | 86.4 | Diluent | |
| Acrylates/ | 0.80 | Rheology | Carbopol ® Ultrez 21 |
| C10-C30 Alkyl | | Modifier | Polymer (Noveon) |
| Acrylates Cross- | | | |
| polymer | | | |
| DMDM Hydan- | 0.30 | Preservative | Glydant ® (Lonza) |
| toin | | | |
| Sodium Hydrox- | 0.60 | Neutralizing | |
| ide (18%) | | Agent | |
| Part B | | | |
| Deionized Water | 6.60 | Diluent | |
| Dimethicone | 1.00 | Conditioner/ | Ultrasil ™ CA-2 Silicone |
| PEG-7 Succinate | | Compat- | |
| | | ibilizing | |
| | | Agent | |
| Aloe Vera Gel | 2.50 | Moisturizer | Aloe Vera Gel Decolor- |
| (40:1) | | | ized, 40X (Terry Labs) |
| Sodium Hydrox- | 0.50 | Neutralizing | |
| ide (18%) | (QS to | Agent | |
| | pH 6.9-7.1) | | |
| Part C | | | |
| Sodium Hydrox- | 0.50 | Neutralizing | |
| ide (18%) | (QS to | Agent | |
| | pH 6.5-6.7) | | |

Procedure:
Part A was prepared - Carbopol ® Ultrez 21 polymer was added to
water and allowed to wet. Glydant was added. Neutralizer was added.
Part B was blended. Part B was added to Part A. Mixture was neutralized

| | |
|---|---|
| Properties | |
| pH | 6.6 |
| Viscosity @ 20 rpm, 20 C. (mPa∃s) | 12,750 |
| Turbidity on Micro 1000 Turbidimeter | 4.3 |
| (NTU) | |
| Stability | Passed 3 months accelerated, |
| | 45° C. |

When preparation of the preceding formulation was repeated without dimethicone copolyol succinate, the final product viscosity was higher (18,200 mPa.s), but the turbidity (15.5 NTU) was not optimal.

### Data for Figures

Data for FIG. 1.

| Percent Ultrasil ™ | | | |
|---|---|---|---|
| CA-1 Silicone | Viscosity (mPa · s) | Turbidity (NTU) | Precipitate |
| 0 | 12,200 | 487 | Yes |
| 0.3 | 13,500 | 120 | Yes |
| 0.5 | 15,200 | 13.1 | No |
| 1.0 | 16,000 | 11.9 | No |

Mucilage viscosity and turbidity is 13,700 mPa · s and 3.8 NTU, respectively.

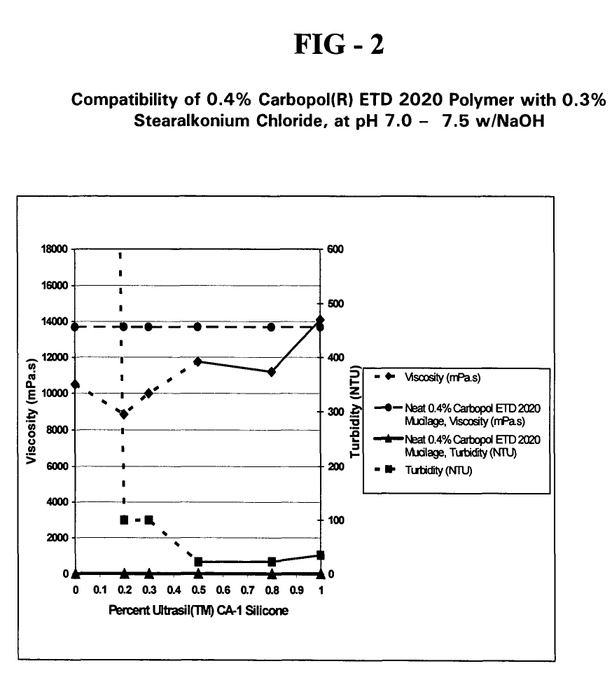

Data for FIG. 2.

| Percent Ultrasil ™ | | | |
|---|---|---|---|
| CA-1 Silicone | Viscosity (mPa · s) | Turbidity (NTU) | Precipitate |
| 0 | 10,500 | >10,000 | Yes |
| 0.2 | 8,850 | 99.5 | Yes |
| 0.3 | 10,000 | 99.7 | Yes |
| 0.5 | 11,750 | 22.1 | No |
| 0.8 | 11,250 | 22.6 | No |
| 1.0 | 14,100 | 34.7 | No |

Mucilage viscosity and turbidity is 13,700 mPa · s and 3.8 NTU, respectively.

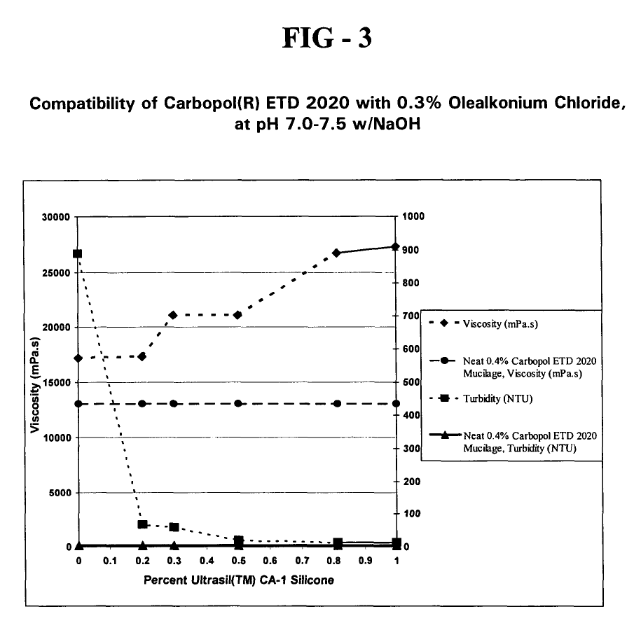

Data for FIG. 3.

| Percent Ultrasil ™ | | | |
|---|---|---|---|
| CA-1 Silicone | Viscosity (mPa · s) | Turbidity (NTU) | Precipitate |
| 0 | 17200 | 889 | Yes |
| 0.2 | 17300 | 66.3 | Yes |
| 0.3 | 21100 | 58.1 | Yes |
| 0.5 | 21050 | 19.5 | Yes |
| 0.8 | 26700 | 12.9 | No |
| 1.0 | 27300 | 13.0 | No |

Mucilage viscosity and turbidity is 13,000 mPa · s and 4.8 NTU, respectively.

Data for FIG. 4.

| Percent DMC | | | |
|---|---|---|---|
| Succinate | Viscosity (mPa · s) | Turbidity (NTU) | Precipitate |
| 0 | 8,180 | >10,000 | Yes |
| 0.2 | 7,160 | >10,000 | Yes |
| 0.3 | 6,220 | >10,000 | Yes |
| 0.5 | 6,160 | 1083 | Yes |
| 0.8 | 4,500 | 811 | No |
| 1.0 | 4,200 | 202 | No |

Mucilage viscosity and turbidity is 31,800 mPa · s and 7.9 NTU, respectively.

Data for FIG. 5.

| Percent | Viscosity (mPa · s) | Turbidity (NTU) | Precipitate |
|---|---|---|---|
| 0 | 8700 | 1159 | Yes |
| 0.2 | 10200 | 325 | Yes |
| 0.3 | 10200 | 198 | Yes |
| 0.5 | 10350 | 75.1 | Yes |
| 0.8 | 11200 | 39.4 | Yes |
| 1.0 | 11050 | 30.1 | Yes |
| 1.5 | 12000 | 26.9 | Yes |
| 2.0 | 12150 | 27.2 | Yes |
| 3.0 | 12100 | 27.6 | No |
| 3.5 | 11400 | 27.6 | No |
| 4.0 | 12100 | 22.8 | No |

Mucilage viscosity and turbidity is 31,900 mPa · s and 3.4 NTU, respectively.

Data for FIG. 6.

| Percent | Viscosity (mPa · s) | Turbidity (NTU) | Precipitate |
|---|---|---|---|
| 0 | 1,500 | >10,000 | No |
| 0.2 | 1,020 | 2,112 | No |
| 0.3 | 1,090 | 1,351 | No |
| 0.5 | 1,010 | 891 | No |
| 0.8 | 1,010 | 34.5 | No |
| 1.0 | 1,090 | 21.8 | No |

Mucilage viscosity and turbidity is 3,670 mPa · s and 10.6 NTU, respectively.

Data for FIG. 7.

| | | | 1% Cetrimonium | 1% Cetromonium |
|---|---|---|---|---|
| | | 1% | Chloride, 1.67% | Chloride, |
| | | Cetrimonium | Dimethicone | 1.67% Dimethicone |
| | Control | Chloride | Copolyol | Copolyol Phthalate |
| Tress 1 | 17.63 | 25.51 | 23.58 | 23.57 |
| Tress 2 | 20.94 | 23.76 | — | 23.02 |
| Avg. | 19.3 | 24.6 | 23.6 | 23.3 |

Data for FIG. 8.

| | | | | 1% |
|---|---|---|---|---|
| | | | 1% | Stearalkonium |
| | | | Stearalkonium | Chloride, |
| | | | Chloride, | 1.67% |
| | | 1% | 1.67% | Dimethicone |
| | | Stearalkonium | Dimethicone | Copolyol |
| | Control | Chloride | Copolyol | Phthalate |
| Tress 1 | 17.63 | 30.87 | 25.99 | 28.45 |
| Tress 2 | 20.94 | 31.50 | — | 27.13 |
| Avg. | 19.3 | 31.2 | 26.0 | 27.8 |

TABLE IX
Data for FIG. 9.

| | | | | 1% |
|---|---|---|---|---|
| | | | 1% | Olealkonium |
| | | | Olealkonium | Chloride, |
| | | | Chloride, | 1.67% |
| | | 1% | 1.67% | Dimethicone |
| | | Olealkonium | Dimethicone | Copolyol |
| | Control | Chloride | Copolyol | Phthalate |
| Tress 1 | 14.94 | 24.87 | 26.60 | 24.66 |
| Tress 2 | 14.59 | 24.95 | 24.22 | 23.82 |
| Avg. | 14.8 | 24.9 | 25.4 | 24.2 |

Data for FIG. 10. All values of work in g · cm.

| | | 1% Cetrimonium | 1% Cetrimonium Chloride, |
|---|---|---|---|
| | 1% Cetrimonium | Chloride, 1.67% | 1.67% Dimethicone |
| | Chloride | Dimethicone Copolyol | Copolyol Phthalate |
| Unconditioned | | | |
| Tress 1 | 2,467 | 3,577 | 3,682 |
| | 1,508 | 1,524 | 2,054 |
| | 1,280 | 1,396 | 1,402 |
| | 1,375 | 1,026 | 1,004 |
| | 891 | 1,111 | 1,163 |
| (Total) | 7,521 | 8,634 | 9,305 |
| Tress 2 | 2,792 | 2,160 | 4,177 |
| | 1,150 | 1,007 | 1,312 |
| | 730 | 898 | 906 |
| | 891 | 596 | 826 |
| | 1,226 | 578 | 606 |
| (Total) | 6,789 | 5,234 | 7,827 |
| Tress 3 | 7,639 | 2,645 | 6,465 |
| | 5,598 | 1,128 | 1,765 |
| | 2,803 | 1,859 | 1,680 |
| | 1,791 | 1,308 | 1,062 |
| | 1,502 | 1,083 | 1,295 |
| (Total) | 19,333 | 4,856 | 12,267 |
| Conditioned | | | |
| Tress 1 | 1,435 | 909 | 1,191 |
| | 1,503 | 759 | 553 |
| | 1,021 | 651 | 654 |
| | 997 | 552 | 542 |
| | 1,016 | 768 | 473 |
| (Total) | 5,972 | 3,639 | 3,413 |
| % Con. | (79.4) | (42.1) | (36.7) |
| Tress 2 | 1,598 | 817 | 604 |
| | 822 | 605 | 538 |
| | 766 | 423 | 395 |
| | 789 | 497 | 354 |
| | 639 | 455 | 338 |
| (Total) | 4,614 | 2,797 | 2,229 |
| % Con. | (68.0) | (53.3) | (28.5) |
| Tress 3 | 3,186 | 1227 | 541 |
| | 1,912 | 612 | 481 |
| | 1,662 | — | 574 |
| | 1,240 | — | 531 |
| | 872 | 466 | 503 |
| (Total) | 8,832 | 2,305 | 2,630 |
| % Con. | (45.7) | (47.5) | (21.4) |
| % Con. (Avg) | (64.4) | (47.7) | (28.9) |

Data for FIG. 11. All values of work in g · cm.

| | | | 1% Stearalkonium |
|---|---|---|---|
| | | 1% Stearalkonium | Chloride, 1.67% |
| | Stearalkonium | Chloride, 1.67% | Dimethicone Copolyol |
| | Chloride, 1% | Dimethicone Copolyol | Phthalate |
| Unconditioned | | | |
| Tress 1 | 3,201 | 2,823 | 1906 |
| | 1,134 | 921 | 1074 |
| | 1,004 | 881 | 833 |
| | 894 | 527 | 1013 |
| | 912 | 654 | 860 |
| (Total) | 7,145 | 5,806 | 5686 |
| Tress 2 | 3,967 | 1,158 | 2011 |
| | 2,812 | 695 | 1142 |
| | 2,758 | 426 | 825 |
| | 1,576 | 769 | 777 |
| | 1,269 | 521 | 592 |
| (Total) | 12,391 | 3,569 | 5347 |
| Tress 3 | 2,471 | 4,314 | 2681 |
| | 819 | 3,740 | 1323 |
| | 943 | 2,317 | 893 |
| | 694 | 1,716 | 846 |
| | 616 | 1,286 | 1156 |
| (Total) | 5,543 | 13,373 | 6899 |
| Conditioned | | | |
| Tress 1 | 1,644 | 1,288 | 1,143 |
| | 1,069 | 859 | 525 |
| | 822 | 415 | 557 |
| | 845 | 450 | 551 |
| | 628 | 384 | 440 |
| (Total) | 5,008 | 3,396 | 3,216 |
| %Con. | (70.1) | (58.5) | (56.6) |
| Tress 2 | 3,854 | 1,019 | 856 |
| | 876 | 560 | 597 |
| | 534 | 909 | 463 |
| | 462 | 770 | 559 |
| | 330 | 634 | 505 |
| (Total) | 6,056 | 3,892 | 2,980 |
| %Con. | (48.9) | (109.1) | (55.7) |
| Tress 3 | 2,826 | 841 | 927 |
| | 1,055 | 611 | 632 |
| | 924 | 489 | 527 |
| | 584 | 639 | 423 |
| | 415 | 583 | 506 |
| (Total) | 5,804 | 3,163 | 3,015 |
| %Con. | (104.7) | (23.7) | (43.7) |
| % Con. (Avg). | (74.6) | (63.8) | (52.0) |

What is claimed is:

1. A method of compatibilizing an anionic polymeric rheology modifier with cationic materials which method comprises complexing the cationic materials with an anionic complexing agent prior to combining the rheology modifier with the complexed cationic material wherein said anionic complexing agent contains a bulky molecule having an anionic group.

2. A method of claim 1, wherein said bulky molecule in a complexing agent has a molecular weight of at least 1,000.

3. A method of claim 2, wherein said bulky molecule is a polymer.

4. A method of claim 3, wherein said polymeric complexing agent is selected from the group consisting of an acrylic copolymer, polyalkylene glycol, polyvinyl alcohol, polyvinyl acetate, polysaccharide, polyurethane and polysilicones.

5. A method of claim 4, wherein said polymeric complexing agents contain an anionic group selected from carboxylate, sulfonate, sulfate, phosphate and phosphonate groups.

6. A method of claim 5, wherein said polymeric complexing agent is a polysilicone.

7. A method of claim 6, wherein said polysilicone is selected from the structure consisting of: 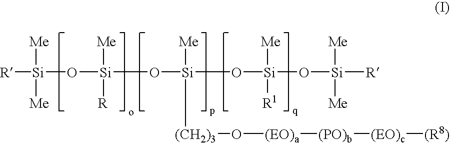
wherein:
Me is methyl;
R and R′ are independently selected from methyl, —OH, —R⁷, and —R⁹-A or —(CH₂)₃—O-(EO)ₐ—(PO)_{b}-(EO)_{c}-G with the proviso that both R and R′ are not methyl, —OH or R⁷;
R′ is selected from lower alkyl CH₃(CH₂)ₙ— or phenyl where n is an integer from 0 to 22;
a, b, and c are integers independently ranging from 0 to 100; 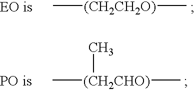
o is an integer ranging from 1 to 200;
q is an integer ranging from 0 to 1000;
p is an integer ranging from 0 to 200;
R⁷ is aryl, alkyl, aralkyl, alkaryl, or alkenyl group of 1-40 carbons;
R⁸ is hydrogen or R⁷ or C(O)—X wherein X is aryl, alkyl, aralkyl, alkaryl, alkenyl group of 1-40 carbons, or a mixture thereof;
R⁹ is divalent group selected from alkylene of 1-40 carbons which may be interrupted with arylene group of 6 to 18 carbons or an alkylene group containing unsaturation of 2 to 8 carbons;
A and G are independently are selected from 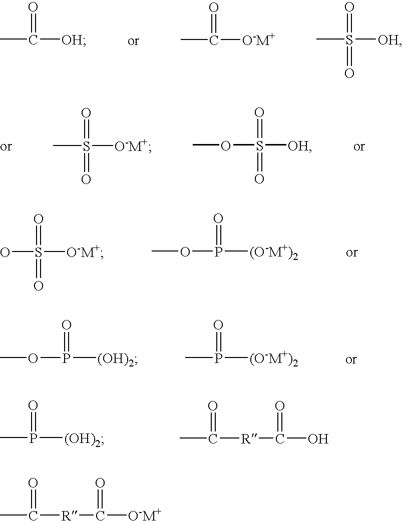
where
R″ is a divalent group selected from alkylene of 1-40 carbons which may be interrupted with an arylene group of 6 to 18 carbons or an alkylene group of 2 to 8 carbons, and is preferably selected from the 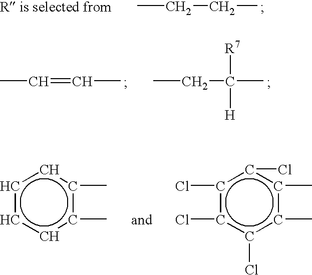
where M is Na, K, Li, NH₄; or an amine containing alkyl, aryl, akenyl, hydroxyalkyl, arylalkyl or alkaryl groups; 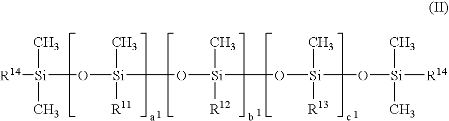
wherein
R¹¹ is selected from lower alkyl having one to eight carbon atoms or phenyl,
R¹² is
—(CH₂)₃—O-(EO)ₓ—(PO)_{y}-(EO)_{z}—SO₃⁻M⁺
M is a cation and is selected from Na, K, Li, or NH₄;
x, y and z are integers independently ranging from 0 to 100;
R¹³ is
—(CH₂)₃—O-(EO)ₓ—(PO)_{y}-(EO)_{z}—H
R¹⁴ is methyl or hydroxyl;
a¹ and c¹ are independently integers ranging from 0 to 50;
b¹ is an integer ranging from 1 to 50; 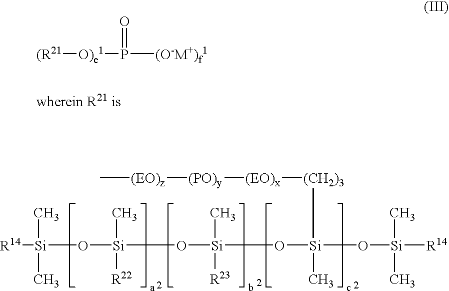
a² is an integer from 0 to 200;
b² is an integer from 0 to 200;
c² is an integer from 1 to 200;
R¹⁴ is as defined above;
R²² is selected from —(CH₂)ₙCH₃ and phenyl;
n is an integer from 0 to 10;
R²³ is —(CH₂)₃—O-(EO)ₓ₁—(PO)_{y1}-(EO)_{z1}—H;
x¹, y¹ ands z¹ are integers and are independently selected from 0 to 20;
e¹ and f¹ are 1 or 2 with the proviso that e+f=3;
M is selected from H, Na, K, Li, or NH₄; and 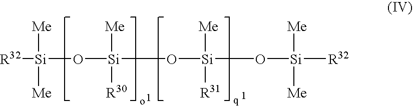
wherein;
Me is methyl;
R³⁰ and R³² independently are —CH₃ or
—(CH₂)₃—O-(EO)ₐ_{³}—(PO)_{b}_{³}-(EO)_{c}_{³}—C(O)—R³³—C(O)—OH;
with the proviso that both R³⁰ and R³² are not —CH₃;
R³³ is selected from —CH₂—CH₂—; —CH═CH—; —CH₂—C(R³⁷)—H; 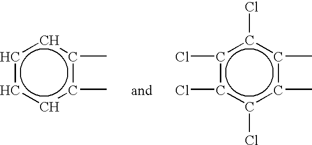
R³⁷ is alkyl having from 1 to 22 carbon atoms;
R³¹ is selected from lower alkyl (having 1-4 carbons), CH₃(CH)ₙ¹— and phenyl;
n¹ is an integer from 0 to 8;
a³, b³ and c³ are integers independently ranging from 0 to 20;
EO is an ethylene oxide residue —(CH₂CH₂—O)—;
PO is a propylene oxide residue —(CH₂CH(CH₃)—O);
o¹ is an integer ranging from 1 to 200;
q¹ is an integer ranging from 0 to 500.

8. A method of claim 5, wherein the anionic rheology modifier is a polymer prepared from ethylenically unsaturated monomers at least 10% by weight of which is a monomer containing carboxylic group.

9. A method of claim 8, wherein said polymer contains at least 25% by weight of repeating units derived from a monomer containing carboxylic group.

10. A method of claim 9, wherein the rheology modifier anionic polymer is selected from the group consisting of
(A) a polymer obtained from the polymerization of one or more monomers represented by the formula 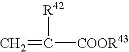
wherein R⁴³ is hydrogen or an alkyl group having from 8 to 30 carbon atoms and
R⁴² is a substituent selected from the class consisting of hydrogen, halogen, hydroxyl, lactone, lactam and the cyanogens (—CN) groups, monovalent alkyl radicals, monovalent aryl radicals, monovalent aralkyl radicals, monovalent alkaryl radicals and monovalent cycloaliphatic radicals; and
(B) a crosslinked copolymer obtained from the copolymerization of a monomeric system comprising:
a) from about 10 to about 97% by weight of at least one ethylenically unsaturated mono- or dicarboxylic acid;
b) from 0 to about 80% by weight of at least one (C₁-C₃₀) alkyl or aralkyl ester of an ethylenically unsaturated mono- or dicarboxylic acid;
c) from about 0.5 to about 80% by weight of at least one associative monomer which is an ester of formula
J-O—(CH₂—CHR₂O)ᵣ—(CH₂)ₛ—R₁
wherein
J is an ethylenically unsaturated acrylic residue, optionally containing an additional carboxylic group, wherein, optionally, said additional carboxylic group may be esterified with a (C₁-C₂₀) aliphatic alkyl group;
R₁ is an alkyl, alkphenyl or aralkyl residue having from 1 to 30 carbon atoms;
R₂ is hydrogen, methyl or ethyl;
r is comprised between 0 and 50;
s is comprised between 0 and 30;
d) from 0 to about 20% by weight of at least one ethylenically unsaturated amide;
e) from about 0.2 to about 20% by weight of at least one diester between a polyoxyalkyleneglycol or an emulsifier having at least two free OH-groups and an ethylenically unsaturated carboxylic acid, as the crosslinking agent; and
f) from 0 to about 20% by weight of at teat one ethylenically unsaturated sulfonic acid.

11. A method of claim 10, wherein R⁴³ is hydrogen or an alkyl group from 10 to 22 carbon atoms and R⁴² is hydrogen or methyl.

12. A composition of matter comprising a cationic material complexed with an anionic complexing agent that contains a bulky molecule having an anionic group and an anionic polymeric rheology modifier.

13. A composition of claim 12, wherein said bulky molecule is a polymer.

14. A composition of claim 13, wherein said polymeric complexing agent is selected from the group consisting of an acrylic copolymer, polyalkylene glycol, polyvinyl alcohol, polyvinyl acetate, polysaccharide, polyurethane and a polysilicone.

15. A composition of claim 14, wherein said polymeric complexing agent contains an anionic group selected from carboxylate, sulfonate, sulfate, phosphate and phosphonate groups.

16. A composition of claim 15, wherein said polymeric complexing agent is a polysilicone.

17. A composition of claim 16, wherein polysilicone is selected from the structures consisting of 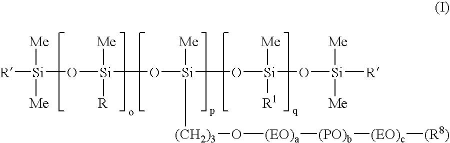
wherein:
Me is methyl;
R and R′ are independently selected from methyl, —OH, —R⁷, and —R⁹-A or —(CH₂)₃—O-(EO)ₐ—(PO)_{b}-(EO)_{c}-G with the proviso that both R and R′ are not methyl, —OH or R⁷;
R¹ is selected from lower alkyl CH₃(CH₂)ₙ— or phenyl where n is an integer from 0 to 22;
a, b, and c are integers independently ranging from 0 to 100; 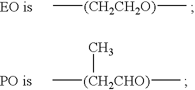
o is an integer ranging from 1 to 200;
q is an integer ranging from 0 to 1000;
p is an integer ranging from 0 to 200;
R⁷ is aryl, alkyl, aralkyl, alkaryl, or alkenyl group of 1-40 carbons;
R⁸ is hydrogen or R⁷ or C(O)—X wherein X is aryl, alkyl, aralkyl, alkaryl, alkenyl group of 1-40 carbons, or a mixture thereof;
R⁹ is divalent group selected from alkylene of 1-40 carbons which may be interrupted with arylene group of 6 to 18 carbons or an alkylene group containing unsaturation of 2 to 8 carbons;
A and G are independently are selected from 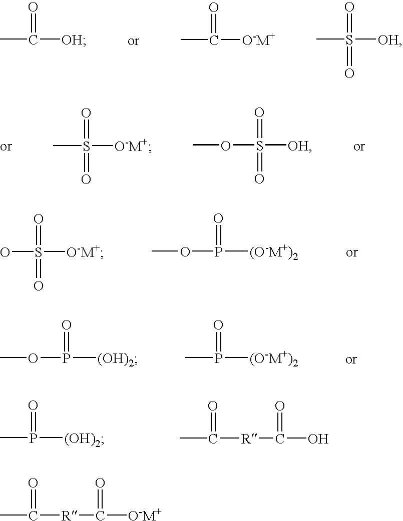
where
R″ is a divalent group selected from alkylene of 1-40 carbons which may be interrupted with an arylene group of 6 to 18 carbons or an alkylene group of 2 to 8 carbons, and is preferably selected from the 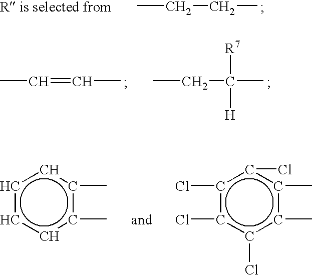
where M is Na, K, Li, NH₄; or an amine containing alkyl, aryl, akenyl, hydroxyalkyl, arylalkyl or alkaryl groups. 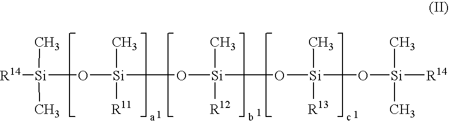
wherein
R¹¹ is selected from lower alkyl having one to eight carbon atoms or phenyl,
R¹² is
—(CH₂)₃—O-EO)ₓ—(PO)_{y}-(EO)_{z}—SO₃⁻M⁺
M is a cation and is selected from Na, K, Li, or NH₄;
x, y and z are integers independently ranging from 0 to 100;
R¹³ is
—(CH₂)₃—O-(EO)ₓ—(PO)_{y}-(EO)_{z}—H
R¹⁴ is methyl or hydroxyl;
a¹ and c¹ are independently integers ranging from 0 to 50;
b¹ is an integer ranging from 1 to 50; 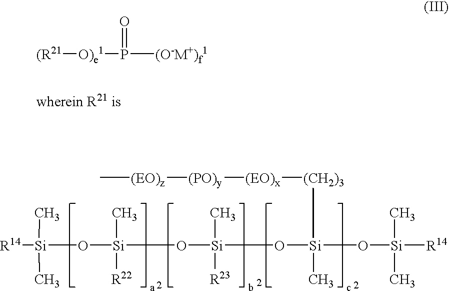
a2 is an integer from 0 to 200;
b² is an integer from 0 to 200;
c²is an integer from 1 to 200;
R¹⁴ is as defined above;
R²² is selected from —(CH₂)ₙCH₃ and phenyl;
n is an integer from 0 to 10;
R²³ is —CH₂)₃—O-(EO)ₓ₁—(PO)_{y1}-(EO)_{z1}—H;
x¹, y¹ ands z¹ are integers and are independently selected from 0 to 20;
e¹ and f¹ are 1 or 2 with the proviso that e+f=3;
M is selected from H, Na, K, Li, or NH₄; and 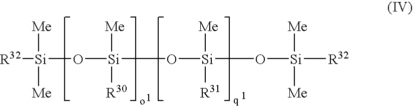
wherein;
Me is methyl;
R³⁰ and R³² independently are CH₃ or
—(CH₂)₃—O-(EO)ₐ_{³}—(PO)_{b}_{³}-(EO)_{c}_{³}—C(O)—R³³—C(O)—OH;
with the proviso that both R³⁰ and R³² are not —CH₃;
R³³ is selected from —CH₂—CH₂—; —CH═CH—; —CH₂—C(R³⁷)—H; 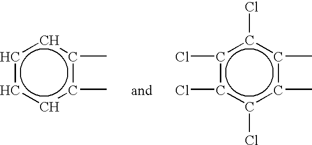
R³⁷ is alkyl having from 1 to 22 carbon atoms;
R³¹ is selected from lower alkyl (having 1-4 carbons), CH₃(CH)ₙ¹— and phenyl;
n¹ is an integer from 0 to 8;
a³, b³ and c³ are integers independently ranging from 0 to 20;
EO is an ethylene oxide residue —(CH₂CH₂—O)—;
PO is a propylene oxide residue —(CH₂CH(CH₃)—O);
o¹ is an integer ranging from 1 to 200;
q¹ is an integer ranging from 0 to 500.

18. A composition of claim 17, wherein the anionic rheology modifier is selected from the group consisting of
(A) a polymer obtained from the polymerization of one or more monomers represented by the formula 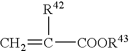
wherein R⁴³ is hydrogen or an alkyl group having from 8 to 30 carbon atoms and
R⁴² is a substituent selected from the class consisting of hydrogen, halogen, hydroxyl, lactone, lactam and the cyanogens (—CN) groups, monovalent alkyl radicals, monovalent aryl radicals, monovalent aralkyl radicals, monovalent alkaryl radicals and monovalent cycloaliphatic radicals; and
(B) a crosslinked copolymer obtained from the copolymerization of a monomeric system comprising:
a) from about 10 to about 97% by weight of at least one ethylenically unsaturated mono- or dicarboxylic acid;
b) from 0 to about 80% by weight of at least one (C₁-C₃₀) alkyl or aralkyl ester of an ethylenically unsaturated mono- or dicarboxylic acid;
c) from about 0.5 to about 80% by weight of at least one associative monomer which is an ester of formula
J-O—(CH₂—CHR₂O)ᵣ—(CH₂)ₛ—R₁
wherein
J is an ethylenically unsaturated acrylic residue, optionally containing an additional carboxylic group, wherein, optionally, said additional carboxylic group may be esterified with a (C₁-C₂₀) aliphatic alkyl group;
R¹ is an alkyl, alkphenyl or aralkyl residue having from 1 to 30 carbon atoms;
R₂ is hydrogen, methyl or ethyl;
r is comprised between 0 and 50;
s is comprised between 0 and 30;
d) from 0 to about 20% by weight of at least one ethylenically unsaturated amide;
e) from about 0.2 to about 20% by weight of at least one diester between a polyoxyalkyleneglycol or an emulsifier having at least two free OH-groups and an ethylenically unsaturated carboxylic acid, as the crosslinking agent; and
f) from 0 to about 20% by weight of at least one ethylenically unsaturated sulfonic acid.

19. A composition of claim 18, wherein R⁴³ is hydrogen or an alkyl group from 10 to 22 carbon atoms and R⁴² is hydrogen or methyl.

20. A hair conditioner comprising a composition of claim 16.

21. A hair conditioner of claim 20 which is a crystal clear formula.

22. A hair conditioner of claim 20 which is a clear styling gel formula.

23. A skin moisturizer comprising a composition of claim 16.

24. A hair shampoo comprising a composition of claim 16.

25. A household product comprising a composition of claim 16.

26. A hand sanitizing gel comprising a composition of claim 16.
